SEQUENCE LISTING (23 sequences)

SEQ ID NO: 1 - DNA, 20 bp, synthetic construct
  source 1..20: mol_type = other DNA
ccgctcaggttctgctttta

SEQ ID NO: 2 - DNA, 20 bp, synthetic construct
  source 1..20: mol_type = other DNA
tggaaggacttgagggactc

SEQ ID NO: 3 - DNA, 21 bp, synthetic construct
  source 1..21: mol_type = other DNA
gaccctggtctgcactctatc

SEQ ID NO: 4 - DNA, 21 bp, synthetic construct
  source 1..21: mol_type = other DNA
tggaagactagtgattttgtt

SEQ ID NO: 5 - DNA, 64 bp, synthetic construct
  source 1..64: mol_type = other DNA
tgctggaccctggtctgcactctatcgttttggccactgactgacgatagagtagaccagggtc

SEQ ID NO: 6 - DNA, 64 bp, synthetic construct
  source 1..64: mol_type = other DNA
cctggaccctggtctactctatcgtcagtcagtggccaaaacgatagagtgcagaccagggtcc

SEQ ID NO: 7 - DNA, 64 bp, synthetic construct
  source 1..64: mol_type = other DNA
tgctgtggaagactagtgattttgttgttttggccactgactgacaacaaaatctagtcttcca

SEQ ID NO: 8 - DNA, 64 bp, synthetic construct
  source 1..64: mol_type = other DNA
cctgtggaagactagattttgttgtcagtcagtggccaaaacaacaaaatcactagtcttccac

SEQ ID NO: 9 - DNA, 20 bp, synthetic construct
  source 1..20: mol_type = other DNA
  modified_base 1: mod_base = OTHER; note = fluorescent dye TYE 563
ctgctgctgctgctgctgct

SEQ ID NO: 10 - RNA, 23 nt, synthetic construct
  source 1..23: mol_type = other RNA
taccctgtagatccgaatttgtg

SEQ ID NO: 11 - RNA, 22 nt, synthetic construct
  source 1..22: mol_type = other RNA
agaccctggtctgcactctatc

SEQ ID NO: 12 - RNA, 21 nt, synthetic construct
  source 1..21: mol_type = other RNA
gcccctgggcctatcctagaa

SEQ ID NO: 13 - RNA, 22 nt, synthetic construct
  source 1..22: mol_type = other RNA
tgaggtagtaggttgtgtggtt

SEQ ID NO: 14 - RNA, 22 nt, synthetic construct
  source 1..22: mol_type = other RNA
agttcttcagtggcaagcttta

SEQ ID NO: 15 - RNA, 24 nt, synthetic construct
  source 1..24: mol_type = other RNA
tggaagactagtgattttgttgtt

SEQ ID NO: 16 - DNA, 19 bp, synthetic construct
  source 1..19: mol_type = other DNA
tgcccctagagacgaccaa

SEQ ID NO: 17 - DNA, 22 bp, synthetic construct
  source 1..22: mol_type = other DNA
gtagttgagtccatacgtgctg

SEQ ID NO: 18 - DNA, 20 bp, synthetic construct
  source 1..20: mol_type = other DNA
tagactactacggccgcaag

SEQ ID NO: 19 - DNA, 20 bp, synthetic construct
  source 1..20: mol_type = other DNA
agctcctccctatgtcgttg

SEQ ID NO: 20 - DNA, 18 bp, synthetic construct
  source 1..18: mol_type = other DNA
aaaaacagcagcaaaagc

SEQ ID NO: 21 - DNA, 17 bp, synthetic construct
  source 1..17: mol_type = other DNA
tctgtcctgataggtcc

SEQ ID NO: 22 - DNA, 20 bp, synthetic construct
  source 1..20: mol_type = other DNA
atgtgcaaggccggtttcgc

SEQ ID NO: 23 - DNA, 20 bp, synthetic construct
  source 1..20: mol_type = other DNA
cgacacgcagctcattgtag

(19) United States
(12) Patent Application Publication
(10) Pub. No.: US 2026/0183276 A1
(43) Pub. Date: Jul. 2, 2026

(54) miRNA DYSREGULATION CORRECTION AS A STRATEGY TO TREAT HUNTINGTON'S DISEASE

(71) Applicant: The Chinese University of Hong Kong, Hong Kong (CN)

(72) Inventors: Zhefan Stephen CHEN, Hong Kong (CN); Ho Yin Edwin CHAN, Hong Kong (CN); Shaohong Isaac PENG, Hong Kong (CN); Lok I Maggie LEONG, Hong Kong (CN)

(21) Appl. No.: 19/130,639

(22) PCT Filed: Nov. 17, 2023
(86) PCT No.: PCT/IB2023/000721

§ 371 Date: May 16, 2025

Related U.S. Application Data
(60) Provisional application No. 63/384,243, filed on Nov. 18, 2022.

(51) Int. Cl. A61K 31/4706; A61P 25/28

(52) U.S. Cl. CPC A61K 31/4706; A61P 25/28

(57) ABSTRACT

The subject invention pertains to compositions comprising Poly(A) RNA polymerase D5 (PAPD5) small molecule inhibitors and methods of using said compositions to treat Huntington's Disease (HD). The PAPD5 small molecule inhibitor is, for example, BCH001 and RG7834. The PAPD5 small molecule inhibitor can mitigate the neuronal defects and cell death in HD.

### CROSS-REFERENCE TO RELATED APPLICATION

This application claims the benefit of U.S. Provisional Patent Application Ser. No. 63/384,243, filed Nov. 18, 2022, which is hereby incorporated by reference in its entirety including any tables, figures, or drawings.

### BACKGROUND OF THE INVENTION

Polyglutamine (polyQ) diseases are caused by the abnormal expansion of CAG repeats in the protein-coding region of genes related to the disease. The CAG repeats encode a polyQ tract, which becomes a part of the affected protein (Lieberman et al., 2019). Patients with polyQ diseases often develop progressive neurodegeneration and motor impairment. To date, nine polyQ disorders have been described: spinocerebellar ataxia (SCA) types 1, 2, 6, 7, and 17; Machado-Joseph disease (MJD/SCA3); Huntington's disease (HD); dentatorubral pallidoluysian atrophy; and spinal and bulbar muscular atrophy (Lieberman et al., 2019). The expanded CAG repeats are well-known to exert their pathogenic effects at the protein level, mainly through a gain-of-function effect conferred by the mutant protein containing the polyQ tract (Lieberman et al., 2019). In addition to proteinopathy, mutant RNAs containing the expanded CAG repeats also contribute to cellular dysfunction through a gain-of-function effect (de Mezer et al., 2011; Li et al., 2008). The transcription of expanded CAG triplet repeat sequences located in the untranslated region does not produce polyQ proteins. Li et al. (2018) expressed non-translatable mutant CAG repeat sequences in vivo and observed neurodegeneration. The genomic expansion of CAG repeats causes the transcription of mutant CAG transcripts, which further leads to the production of RNA foci (de Mezer et al., 2011) and polyQ aggregates (Lieberman et al., 2019), and subsequent neurotoxicity.

Several studies have reported that microRNA (miRNA) homeostasis is perturbed in polyQ diseases (Dong and Cong, 2019), including HD (Megret et al., 2020; Olmo et al., 2021; Wang et al., 2020). MicroRNAs (miRNAs) are small non-coding RNAs that are responsible for RNA silencing and the posttranscriptional regulation of gene expression. miRNAs are classified as small non-coding RNAs and they are components of the RNA-induced silencing complex (RISC), which guides the post-transcriptional silencing of mRNA targets (Jonas and Izaurralde, 2015). Supplementing HD disease mice with miRNAs has been shown to be a potential therapeutic strategy (Cheng et al., 2013; Fukuoka et al., 2018). Poly(A) RNA polymerase D5 (PAPD5) is one of the enzymes that target miRNAs for degradation through the addition of adenosine to the 3′ end of the miRNAs, which triggers their 3′-to-5′ trimming and subsequent degradation (Boele et al., 2014; Wyman et al., 2011). PAPD5 is robustly upregulated in polyQ disease models. However, there remains limited treatments for HD or symptoms thereof.

Therefore, there is an urgent need for compounds to treat HD and symptoms thereof.

### BRIEF SUMMARY OF THE INVENTION

The subject invention pertains to compositions comprising Poly(A) RNA polymerase D5 (PAPD5) small molecule inhibitors and methods of using said composition to treat Huntington's Disease (HD). In certain embodiments, the PAPD5 small molecule inhibitor is, for example, BCH001 and RG7834. In certain embodiments, the PAPD5 small molecule inhibitor can mitigate the neuronal defects and cell death in HD.

MicroRNAs (miRNAs) are small non-coding RNAs that are responsible for RNA silencing and the posttranscriptional regulation of gene expression. PAPD5 catalyzes the addition of adenosine to the 3′ end of miRNAs, which promotes their subsequent degradation. Yin Yang 1 (YY1) protein, a transcriptional repressor of PAPD5, was recruited to both RNA foci and protein aggregates, which caused the upregulation of PAPD5 in Huntington's disease (HD). A subset of PAPD5-regulated miRNAs have downregulated levels in the HD model. The reduction of miR-504-5p and miR-7-5p caused the activation of the TAB1/2-mediated TAK1-MKK4-JNK proapoptotic pathway.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1T. Mutant CAG RNA induces PAPD5 expression and activates TAK1MKK4-JNK caspase 3 cascade. (FIG. 1A) Both transcriptional and translational levels of PAPD5 were increased in EGFPCAG78-transfected SK-N-MC cells. (FIG. 1B) Knockdown of PAPD5 suppressed EGFPCAG78-induced cell death. (FIG. 1C) Pseudopupil assay showed that knockdown of PAPD5 restored retinal degeneration in DsRedCAG100 adult Drosophila eyes. Flies of genotypes: w; gmr::Gal4 UAS::DsRed_{CAG0}/+; +/+, w; gmr::Gal4 UAS.:DsRed_{CAG0/+}; UAS::PAPD5-dsRNAGD19799/+, w; gmr::Gal4/+; UAS::DsRedCAG100/+ and w; gmr::Gal4/+; UAS::DsRed_{CAG100}/UAS::PAPD5-dsRNA^{GD19799}. (FIG. 1D) Quantification of (C). (FIG. 1E) RT-PCR analysis of PAPD5 expression in DsRed_{CAG100} flies. Overexpression of PAPD5 induced cleavage of caspase 3 (FIG. 1F) and cell death (FIG. 1G) in a dose-dependent manner. (FIG. 1H) Schematic of MKK-JNK apoptotic pathway. (FIG. 1I) Enhanced phosphorylation level of JNK and MKK4, but not MKK7, was observed in SK-N-MC cells overexpressed with PAPD5. (FIG. 1J) Schematic of how PAPD5 triggers the activation of TAK1 and MKK4. (FIG. 1K) Overexpression of PAPD5 enhanced phosphorylation of TAK1 in a dose-dependent manner. PAPD5-induced TAK1-MKK4JNK pro-apoptotic signaling (FIG. 1L), and cell death (FIG. 1M) could be suppressed by TAK1 inhibitor (5Z-7-oxozeaenol) in a dose-dependent manner. JNK inhibitor (SP600125) treatment diminished JNK, but not TAK1 and MKK4, phosphorylation and cleavage of caspase 3 (FIG. 1N), and cell death (FIG. 1O) in PAPD5-transfected cells in a dose-dependent manner. (FIG. 1P) Knockdown of PAPD5 inhibited EGFP_{CAG78}-induced TAK1-MKK4-JNK phosphorylation and caspase 3 cleavage. Mutant CAG RNA-induced cell death was rescued upon 5Z-7-oxozeaenol (FIG. 1Q) or SP600125 (FIG. 1S) treatment. (FIG. 1R) TAK1 inhibitor treatment suppressed EGFP_{CAG78}-induced TAK1-MKK4-JNK pro-apoptotic signaling. (FIG. 1T) Phosphorylation of JNK and cleavage of caspase 3 were attenuated upon SP600125 treatment in EGFP_{CAG78}-expressing cells. Error bars represent ±S.D. Statistical analysis was performed using one-way ANOVA. NS indicates no significance, *** denotes P<0.001. beta-actin or beta-tubulin was used as loading control. Experiments were independently repeated for three times. Only representative images, gels and blots are shown.

FIGS. 2A-2H. Mutant CAG RNA induces cell death via PAPD5-manipulated miRNA species. (FIG. 2A) Heat map analysis demonstrated the list of 186 miRNAs manipulated directly by PAPD5 in EGFP_{CAG78}-expressing SK-N-MC cells. The fold-change is relative to the untransfected control. (FIG. 2B) Top ten enriched KEGG pathways of the predicted mRNA targets of these 186 miRNAs revealed that different cellular pathways are modulated, including the MAPK signaling pathway. (FIG. 2C) TAB1 is a predicted mRNA target for miR-10a-5p, miR-504-5p and miR-331-3p; while TAB2 is the predicted target for let-7b-5p, miR-22-5p and miR-7-5p. EGFP_{CAG78}-induced cell death was dose dependently rescued upon overexpression of miR-504 (FIG. 2D) or miR-7 (FIG. 2E). Co-transfection of miR-504 and miR-7 synergistically suppressed EGFP_{CAG78}-induced cell death (FIG. 2F) and activation of TAK1-MKK4-JNK pro-apoptotic signaling pathway (FIG. 2G). (FIG. 2H) A schematic illustrating miR-504 and miR-7 synergistically target the PAPD5-mediated TAK1-MKK4-JNK pro-apoptotic signaling pathway. Error bars represent ±S.D. Statistical analysis was performed using one-way ANOVA. NS indicates no significance, *** denotes P<0.001. beta-tubulin was used as loading control. Experiments were independently repeated for three times. Only representative blots are shown.

FIGS. 3A-3M. PAPD5-mediated TAK1-MKK4-JNK pro-apoptotic signaling pathway is activated in HD models. (FIG. 3A) Time-dependent induction of the levels of PAPD5, TAB1, TAB2, phospho-TAK1, phospho-MKK4, phospho-JNK and cleavage of caspase 3 in R6/2 HD transgenic mouse brains. (FIG. 3B) Pseudopupil assay revealed that knockdown of PAPD5 alleviated mutant Htt exon1Q93-induced retinal degeneration in fly eyes. Flies of genotypes: w; gmr::Gal4/+; +/+, w; gmr::Gal4/+; UAS::PAPD5dsRNA^{GD19799}/+, w; gmr::Gal4 UAS::Htt exon1Q93/+; +/+ and w; gmr::Gal4 UAS::Htt exon1Q93/+; UAS::PAPD5-dsRNA^{GD19799}/+. (FIG. 3C) Quantification of (FIG. 3B). (FIG. 3D) RT-PCR analysis of PAPD5 expression in Htt exon1Q93 flies. (FIG. 3E) Both transcriptional and translational levels of PAPD5 were upregulated in mutant EGFP Htt1-550_{CAG89} expressing SK-N-MC cells. Knockdown of PAPD5 suppressed EGFP Htt1-550_{CAG89} induced cell death (FIG. 3F) and phosphorylation of TAK1, MKK4, JNK, as well as cleavage of caspase 3 (FIG. 3G). JNK inhibitor SP600125 treatment attenuated phosphorylation of JNK and cleavage of caspase 3 (FIG. 3H) and subsequent cell death (FIG. 3I) in EGFP Htt1550_{CAG89}-transfected cells. Upon the treatment of TAK1 inhibitor 5Z-7-oxozeaenol, EGFP Htt1-550_{CAG89}-induced phosphorylation of TAK1, MKK4 and JNK, and cleavage of caspase 3 (FIG. 3J) and cell death (FIG. 3K) were rescued. Co-transfection of miR-504 and miR-7 synergistically suppressed EGFP Htt1-550_{CAG89}-induced activation of TAK1MKK4-JNK pro-apoptotic signaling pathway (FIG. 3L) and cell death (FIG. 3M). Error bars represent ±S.D. Statistical analysis was performed using one-way ANOVA. NS indicates no significance, *** denotes P<0.001. beta-actin or beta-tubulin was used as loading control. Experiments were independently repeated for three times. Only representative images, gels and blots are shown.

FIGS. 4A-4D. BCH001 rescues PAPD5-mediated caspase cascade in HD. (FIG. 4A) The PAPD5 inhibitor BCH001 treatment rescued PAPD5-induced cell death in a dose-dependent manner. (FIG. 4B) PAPD5-induced phosphorylation of TAK1, MKK4 and JNK, and cleavage of caspase 3 could be dose-dependently suppressed by BCH001 treatment. The activation of TAK1-MKK4-JNK pro-apoptotic signaling pathway (FIG. 4C) and enhanced cell death (FIG. 4D) detected in EGFP Htt1-550_{CAG89}-expressing cells could be suppressed by BCH001. Error bars represent ±S.D. Statistical analysis was performed using one-way ANOVA. NS indicates no significance, *** denotes P<0.001. beta-tubulin was used as loading control. Experiments were independently repeated for three times. Only representative blots are shown.

FIGS. 5A-5W. BCH001 rescues impaired neurite growth and synapse loss in mouse primary cortical neurons expressing Htt1-550_{CAG89}. (FIG. 5A) Representative staining images of Synapsin I in EGFP Htt1-550_{CAG89}-expressing mouse primary cortical neurons. (FIG. 5B) Quantification of (FIG. 5A). Puncta number of Synapsin I per 100 μm was significantly decreased in EGFP Htt1-550_{CAG89}-expressing neurons. (FIG. 5C) Representative staining images of pre-synaptic marker, Bassoon, in mouse neurons. (FIG. 5D) Quantification of (FIG. 5C). Expression of mutant EGFP Htt1-550_{CAG89} dramatically reduced Bassoon puncta in neurons. (FIG. 5E) Representative staining images of postsynaptic marker, Homer1, in mouse neurons. (FIG. 5F) Quantification of (FIG. 5E). Homer1 expression was significantly reduced upon EGFP Htt1-550_{CAG89} transfection in neurons. Primary (FIG. 5G), secondary (FIG. 5H) and tertiary (FIG. 5I) neurites in EGFP Htt1-550_{CAG89} expressing neurons were significantly diminished. (FIG. 5J) Representative staining images of Synapsin I, Bassoon and Homer1 in EGFP Htt1-550_{CAG89}-expressing neurons upon treatment of BCH001. (FIGS. 5K-5M) Quantification of (FIG. 5J). BCH001 treatment restored puncta number of Synapsin I, Bassoon and Homer1 in EGFP Htt1-550_{CAG89}-transfected neurons. The diminished primary (FIG. 5N), secondary (FIG. 5O) and tertiary (FIG. 5P) neurites in EGFP Htt1-550_{CAG89}-transfected neurons were restored upon BCH001 treatment. Ten transfected neurons per replica were used for quantification. Scale bars: 20 sm. (FIG. 5Q) Western blotting analysis on EGFP Htt1-550_{CAG89}-expressing neurons showed that both JNK phosphorylation and caspase 3 cleavage were inhibited by BCH001 treatment. Error bars represent ±S.D. Statistical analysis was performed using one-way ANOVA. NS indicates no significance, *** denotes P<0.001. beta-tubulin was used as loading control. Experiments were independently repeated for three times. (FIGS. 5R-5S) BCH001 treatment restores retinal degeneration in in vivo Drosophila model of Huntington's Disease. (FIGS. 5T-5U) Treatment of A-beta1-42 peptide transgenic model of Alzheimer's Disease with BCH001 and another PAPD5 inhibitor RG7834 both restored neurodegeneration. (FIGS. 5V-5W) Knockdown of dPAPD5 gene using 3 individual dsRNA fly lines GD19799, GD41096, and GD45261 in the A-beta1-42 peptide-expressing Alzheimer's Disease transgenic Drosophila model could rescue retinal degeneration. Only representative images and blots are shown.

FIGS. 6A-6L. Yin Yang 1 protein is recruited to CAG RNA foci and polyQ aggregates and its overexpression de-represses PAPD5 expression. (FIG. 6A) A putative YY1 binding site (TGATGG) within the PAPD5^{+561/+860} promoter region is conserved in mammals. (FIG. 6B) Luciferase reporter analysis showed that a single point mutation in the YY1 binding site within the PAPD5^{+561/+860} promoter region increased luciferase activity. (FIG. 6C) Knockdown of YY1 significantly elevated PAPD5 expression at both transcriptional and translational levels. (FIG. 6D) Luciferase activity of wild-type PAPD5^{+561/+860} promoter was increased in EGFP_{CAG78}-expressing cells. Overexpression of YY1 restored PAPD5 expression at both transcriptional and translational levels in EGFP_{CAG78}- (FIG. 6E) and EGFP Htt1-550_{CAG89}-expressing (FIG. 6F) cells. (FIG. 6G) Immunofluorescence analysis showed that both CAG RNA foci and polyQ aggregates were detected in EGFP Htt1-550_{CAG89}-expressing cells. YY1 protein was found to co-localize with CAG RNA foci and polyQ aggregates. Scale bars: 10 μm. (FIGS. 6H-6J) Quantification of (FIG. 6G). (FIG. 6K) Soluble protein level of YY1 decreased in EGFP Htt1550_{CAG89}-transfected cells. (FIG. 6L) Progressive decrease of soluble YY1 protein level was detected in R6/2 HD mouse brains. Error bars represent ±S.D. Statistical analysis was performed using one-way ANOVA. NS indicates no significance, ** denotes P<0.01, *** denotes P<0.001. beta-actin or beta-tubulin was used as loading control. Experiments were independently repeated for three times. Only representative images, gels and blots are shown.

FIG. 7. A proposed model for PAPD5-mediated TAK1-MKK4-JNK pro-apoptotic signaling pathway in polyQ diseases. In polyQ diseases, including HD, YY1 protein is recruited by CAG RNA foci and polyQ aggregates and this results in the de-repression of PAPD5 transcription. Upregulation of PAPD5 expression leads to the degradation of miR-504 and miR-7, and triggers TAK1-MKK4-JNK caspase cascade, and eventually results in synaptic defects and neuronal cell death.

FIGS. 8A-8B. Knockdown of PADP5 expression did not rescue the external eye phenotypes induced by mutant CGG- or CUG-repeat RNA expression in Drosophila. Knockdown of PAPD5 expression did not alter the external eye phenotypes induced by transgenic expression of CGG₉₀ (FIG. 8A) and CTG₄₈₀ (FIG. 8B) in Drosophila. Flies of genotypes: w; gmr::Gal4/+; +/+, w; gmr::Gal4/+; UAS::PAPD5dsRNA^{GD19799}/+, w; gmr::Gal4 UAS::EGFP-CGG₉₀/+; +/+, w; gmr::Gal4 UAS::EGFPCGG₉₀/+; UAS::PAPD5-dsRNA^{GD19799}/+, w; gmr::Gal4/+; UAS::CTG₆₀/+, w; gmr::Gal4/+; UAS::CTG₆₀/UAS::PAPD5-dsRN^{AGD19799}, w; gmr::Gal4/+; UAS::CTG₄₈₀/+ and w; gmr::Gal4/+; UAS::CTG₄₈₀/UAS::PAPD5-dsRNA^{GD19799}. Experiments were independently repeated for three times. Only representative images are shown.

FIGS. 9A-9D. Overexpression of miRNAs suppresses mutant CAG-repeat RNA-induced caspase cascade. (FIG. 9A) Overexpression of miR-10a-5p, miR-504-5p and miR-331-3p were able to silence TAB1 expression and inhibit TAK1-triggered cell death in EGFP_{CAG78}-transfected cells. (FIG. 9B) miR-7-5p, miR-22-5p and let-7b-5p were capable of silencing TAB2 expression and inhibiting EGFP_{CAG78}-induced TAK1 functional deregulation. (FIG. 9C) miR-504 overexpression reduced TAB1 protein level, the phosphorylation of TAK1 and JNK, and cleavage of caspase 3 in EGFP_{CAG78} expressing cells in a dose-dependent manner. (FIG. 9D) EGFP_{CAG78}-induced phosphorylation of TAK1 and JNK and cleavage of caspase 3 could be inhibited by miR-7-mediated downregulation of TAB2 expression. beta-tubulin was used as loading control. Experiments were independently repeated for three times. Only representative blots are shown.

FIGS. 10A-10D. Knockdown of PAPD5 rescues neurodegeneration in a Drosophila model of Machado-Joseph Disease. (FIG. 10A) Both transcriptional and translational levels of PAPD5 were upregulated in trMJD_{CAG78}-expressing cells. (FIG. 10B) Pseudopupil assay revealed that knockdown of PAPD5 expression alleviated mutant MJD-induced retinal degeneration in adult fly eyes. Flies of genotypes: w; gmr::Gal4 UAS::flMJDQ27/+; +/+, w; gmr::Gal4 UAS::flMJDQ27/+; UAS.::PAPD5-dsRNA^{GD19799}/+, w; gmr::Gal4/+; UAS::flMJDQ84/+ and w; gmr::Gal4/+; UAS::flMJDQ84/UAS::PAPD5-dsRNA^{GD19799}. (FIG. 10C) Quantification of (FIG. 10B). (FIG. 10D) RT-PCR analysis of PAPD5 expression in flMJDQ84 flies. Error bars represent ±S.D. Statistical analysis was performed using one-way ANOVA. NS indicates no significance, *** denotes P<0.001. beta-actin or beta-tubulin was used as loading control. Experiments were independently repeated for three times. Only representative images, gels and blots are shown.

FIG. 11. BCH001 rescues mutant CAG RNA-induced cell death. BCH001 suppressed EGFPCAG78-induced cell death with an IC₅₀ value of 14.67 nM. Error bars represent ±S.D. Experiments were independently repeated for three times.

### BRIEF DESCRIPTION OF THE SEQUENCES

SEQ ID NO: 1: genotyping primer
SEQ ID NO: 2: genotyping primer
SEQ ID NO: 3: a miR-504 mimic sequence
SEQ ID NO: 4: a miR-7 mimic sequence
SEQ ID NO: 5: : miR-504 forward
SEQ ID NO: 6: : miR-504 reverse
SEQ ID NO: 7: : miR-7 forward
SEQ ID NO: 8: : miR-7 reverse
SEQ ID NO: 9: TYE563-labeled Affinity Plus DNA probe
SEQ ID NO: 10: miR-10a-5p
SEQ ID NO: 11: miR-504-5p
SEQ ID NO: 12: miR-331-3p
SEQ ID NO: 13: let-7b-5p
SEQ ID NO: 14: miR-22-5p
SEQ ID NO: 15: miR-7-5p
SEQ ID NO: 16: human PAPD5-forward primer
SEQ ID NO: 17: human PAPD5-reverse primer
SEQ ID NO: 18: fly PAPD5-forward primer
SEQ ID NO: 19: fly PAPD5-reverse primer
SEQ ID NO: 20: CAG-forward primer
SEQ ID NO: 21: CAG-reverse primer
SEQ ID NO: 22: β-actin-forward primer
SEQ ID NO: 23: β-actin-reverse primer

### DETAILED DISCLOSURE OF THE INVENTION

### Selected Definitions

As used herein, the singular forms “a,” “an,” and “the” are intended to include the plural forms as well, unless the context clearly indicates otherwise. Further, to the extent that the terms “including,” “includes,” “having,” “has,” “with,” or variants thereof are used in either the detailed description and/or the claims, such terms are intended to be inclusive in a manner similar to the term “comprising.” The transitional terms/phrases (and any grammatical variations thereof) “comprising,” “comprises,” “comprise,” include the phrases “consisting essentially of,” “consists essentially of,” “consisting,” and “consists.”

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which will depend in part on how the value is measured or determined, i.e., the limitations of the measurement system. Where particular values are described in the application and claims, unless otherwise stated the term “about” meaning within an acceptable error range for the particular value should be assumed.

In the present disclosure, ranges are stated in shorthand, to avoid having to set out at length and describe each and every value within the range. Any appropriate value within the range can be selected, where appropriate, as the upper value, lower value, or the terminus of the range. For example, a range of 1-10 represents the terminal values of 1 and 10, as well as the intermediate values of 2, 3, 4, 5, 6, 7, 8, 9, and all intermediate ranges encompassed within 1-10, such as 2-5, 2-8, and 7-10. Also, when ranges are used herein, combinations and sub-combinations of ranges (e.g., subranges within the disclosed range) and specific embodiments therein are intended to be explicitly included.

In certain embodiments of the invention a subject is a mammal. Non-limiting examples of a mammal treatable according to the methods of the current invention include mouse, rat, dog, guinea pig, cow, horse, cat, rabbit, pig, monkey, ape, chimpanzee, and human. Additional examples of mammals treatable with the methods of the current invention are well known to a person of ordinary skill in the art and such embodiments are within the purview of the current invention.

For the purposes of this invention the terms “treatment, treating, treat” or equivalents of these terms refer to healing, alleviating, relieving, altering, remedying, ameliorating, improving, or affecting the condition or the symptoms of a subject suffering with a disease or condition, for example, Huntington's Disease (HD). The subject to be treated can be suffering from or at risk of developing the disorder or condition, for example, HD. When provided therapeutically, the compound can be provided before the onset of a symptom. The therapeutic administration of the substance serves to attenuate any actual symptom.

For the purposes of this invention, the terms “preventing, preventive, prophylactic” or equivalents of these terms are indicate that the compounds of the subject invention are provided in advance of any disease symptoms and are a separate aspect of the invention (i.e., an aspect of the invention that is distinct from aspects related to the terms “treatment, treating, treat” or equivalents of these terms which refer to healing, alleviating, relieving, altering, remedying, ameliorating, improving, or affecting the condition or the symptoms of a subject suffering from HD). The prophylactic administration of the compounds of the subject invention serves to prevent, reduce the likelihood, or attenuate one or more subsequent symptoms or condition.

By “therapeutically effective dose,” “therapeutically effective amount”, or “effective amount” is intended to be an amount of a compounds of the subject invention disclosed herein that, when administered to a subject, decreases the number or severity of symptoms or inhibits or eliminates the progression or initiation of HD or reduces any increase in symptoms, or improve the clinical course of the disease as compared to untreated subjects. “Positive therapeutic response” refers to, for example, improving the condition of at least one of the symptoms of HD.

An effective amount of the therapeutic agent is determined based on the intended goal. The term “unit dose” refers to a physically discrete unit suitable for use in a subject, each unit containing a predetermined quantity of the therapeutic composition calculated to produce the desired response in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are particular to each individual. Generally, the dosage of the compounds of the subject invention will vary depending upon such factors as the patient's age, weight, height, sex, general medical condition and previous medical history.

In some embodiments of the invention, the method comprises administration of multiple doses of the compounds of the subject invention. The method may comprise administration of 1, 2, 3, 4, 5, 6, 7, 8, 9, 10, 15, 20, 25, 30, 35, 40, 50, 75, 100, 150, 200, 250, 300, 350, 400, 450, 500, 600, 700, 800, 900, 1000 or more therapeutically effective doses of a composition comprising the compounds of the subject invention as described herein. In some embodiments, doses are administered over the course of 1 day, 2 days, 3 days, 4 days, 5 days, 6 days, 7 days, 10 days, 14 days, 21 days, 30 days, 2 months, 3 months, 6 months, 1 year, 2 years, 3 years, 4 years, 5 years, 6 years, 7 years, 8 years, 9 years, 10 years, or more than 10 years. The frequency and duration of administration of multiple doses of the compositions is such as to inhibit or delay the initiation of HD or reduce the symptoms of HD. Moreover, treatment of a subject with a therapeutically effective amount of the compounds of the invention can include a single treatment or can include a series of treatments. It will also be appreciated that the effective dosage of a compound used for treatment may increase or decrease over the course of a particular treatment. Changes in dosage may result and become apparent from the results of diagnostic methods for detecting a HD or symptoms thereof known in the art. In some embodiments of the invention, the method comprises administration of the compounds at a single time per day or several times per day, including but not limiting to 2 times per day, 3 times per day, and 4 times per day.

As used herein, the term “Huntington's Disease” or “HD” refers to a progressive brain disorder caused by a single defective gene on human chromosome 4. The hallmark symptom of Huntington's disease is uncontrolled movement of the arms, legs, head, face and upper body. Huntington's disease also causes a decline in thinking and reasoning skills, including memory, concentration, judgment, and ability to plan and organize.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

### Compounds

In preferred embodiments, the compositions and methods according to the subject invention utilize isolated Poly(A) RNA polymerase D5 (PAPD5) small molecule inhibitors, including, for example, BCH001 and RG7834. PAPD5 small molecule inhibitors may be added to compositions at concentrations of about 0.1 nM to about 1000 nM, preferably, about 1 nM to about 500 nM, more preferably, about 200 nM to about 400 nM. In another embodiment, purified compounds PAPD5 small molecule inhibitors may be in combination with an acceptable carrier, in that PAPD5 small molecule inhibitors may be presented at concentrations of about 0.1 nM to about 1000 nM, preferably, about 1 nM to about 500 nM, more preferably, about 200 nM to about 400 nM.

The following is a chemical formula of BCH001 (Formula (I)) and RG7834 (Formula (II)):

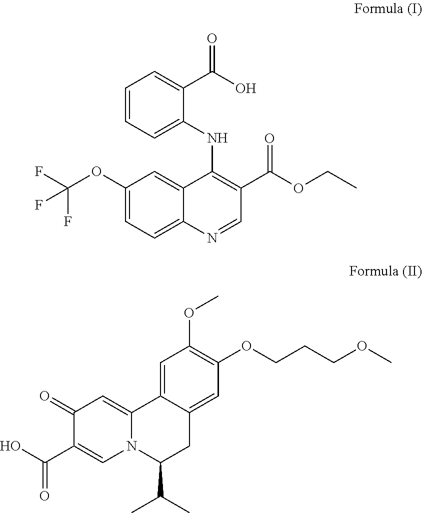 [SMILES: CCOC(=O)c1cnc2ccc(OC(F)(F)F)cc2c1Nc1ccccc1C(=O)O ; COCCCOc1cc2c(cc1OC)-c1cc(=O)c(C(=O)O)cn1[C@H](C(C)C)C2]

In certain embodiments, the PAPD5 small molecule inhibitor compositions can be added to existing compositions that are traditionally used as therapeutics.

In one embodiment, the subject compositions are formulated as an orally-consumable product, such as, for example a food item, capsule, pill, or drinkable liquid. An orally deliverable pharmaceutical is any physiologically active substance delivered via initial absorption in the gastrointestinal tract or into the mucus membranes of the mouth. The topic compositions can also be formulated as a solution that can be administered via, for example, injection, which includes intravenously, intraperitoneally, intramuscularly, intrathecally, intracerebroventricularly or subcutaneously. In other embodiments, the subject compositions are formulated to be administered via the skin through a patch or directly onto the skin for local or systemic effects. The compositions can be administered sublingually, buccally, rectally, or vaginally. Furthermore, the compositions can be sprayed into the nose for absorption through the nasal membrane, nebulized, inhaled via the mouth or nose, or administered in the eye or ear.

Orally consumable products according to the invention are any preparations or compositions suitable for consumption, for nutrition, for oral hygiene, or for pleasure, and are products intended to be introduced into the human or animal oral cavity, to remain there for a certain period of time, and then either be swallowed (e.g., food ready for consumption or pills) or to be removed from the oral cavity again (e.g., chewing gums or products of oral hygiene or medical mouth washes). While an orally-deliverable pharmaceutical can be formulated into an orally consumable product, and an orally consumable product can comprise an orally deliverable pharmaceutical, the two terms are not meant to be used interchangeably herein.

Orally consumable products include all substances or products intended to be ingested by humans or animals in a processed, semi-processed, or unprocessed state. This also includes substances that are added to orally consumable products (particularly food and pharmaceutical products) during their production, treatment, or processing and intended to be introduced into the human or animal oral cavity.

Orally consumable products can also include substances intended to be swallowed by humans or animals and then digested in an unmodified, prepared, or processed state; the orally consumable products according to the invention therefore also include casings, coatings, or other encapsulations that are intended to be swallowed together with the product or for which swallowing is to be anticipated.

In one embodiment, the orally consumable product is a capsule, pill, syrup, emulsion, or liquid suspension containing a desired orally deliverable substance. In one embodiment, the orally consumable product can comprise an orally deliverable substance in powder form, which can be mixed with water or another liquid to produce a drinkable orally-consumable product.

In some embodiments, the orally-consumable product according to the invention can comprise one or more formulations intended for nutrition or pleasure. These particularly include baking products (e.g., bread, dry biscuits, cake, and other pastries), sweets (e.g., chocolates, chocolate bar products, other bar products, fruit gum, coated tablets, hard caramels, toffees and caramels, and chewing gum), alcoholic or non-alcoholic beverages (e.g., cocoa, coffee, green tea, black tea, black or green tea beverages enriched with extracts of green or black tea, Rooibos tea, other herbal teas, fruit-containing lemonades, isotonic beverages, soft drinks, nectars, fruit and vegetable juices, and fruit or vegetable juice preparations), instant beverages (e.g., instant cocoa beverages, instant tea beverages, and instant coffee beverages), meat products (e.g., ham, fresh or raw sausage preparations, and seasoned or marinated fresh meat or salted meat products), eggs or egg products (e.g., dried whole egg, egg white, and egg yolk), cereal products (e.g., breakfast cereals, muesli bars, and pre-cooked instant rice products), dairy products (e.g., whole fat or fat reduced or fat-free milk beverages, rice pudding, yoghurt, kefir, cream cheese, soft cheese, hard cheese, dried milk powder, whey, butter, buttermilk, and partly or wholly hydrolyzed products containing milk proteins), products from soy protein or other soy bean fractions (e.g., soy milk and products prepared thereof, beverages containing isolated or enzymatically treated soy protein, soy flour containing beverages, preparations containing soy lecithin, fermented products such as tofu or tempeh products prepared thereof and mixtures with fruit preparations and, optionally, flavoring substances), fruit preparations (e.g., jams, fruit ice cream, fruit sauces, and fruit fillings), vegetable preparations (e.g., ketchup, sauces, dried vegetables, deep-freeze vegetables, pre-cooked vegetables, and boiled vegetables), snack articles (e.g., baked or fried potato chips (crisps) or potato dough products and extrudates on the basis of maize or peanuts), products on the basis of fat and oil or emulsions thereof (e.g., mayonnaise, remoulade, and dressings), other ready-made meals and soups (e.g., dry soups, instant soups, and pre-cooked soups), seasonings (e.g., sprinkle-on seasonings), sweetener compositions (e.g., tablets, sachets, and other preparations for sweetening or whitening beverages or other food). The present compositions may also serve as semi-finished products for the production of other compositions intended for nutrition or pleasure.

The subject composition can further comprise one or more pharmaceutically acceptable carriers, and/or excipients, and can be formulated into preparations, for example, solid, semi-solid, liquid, or gaseous forms, such as tablets, capsules, powders, granules, ointments, solutions, suppositories, injections, inhalants, and aerosols.

The term “pharmaceutically acceptable” as used herein means compatible with the other ingredients of a pharmaceutical composition and not deleterious to the recipient thereof.

Carriers and/or excipients according the subject invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Polysorbate 65, Polysorbate 80), colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners (e.g. carbomer, gelatin, or sodium alginate), coatings, preservatives (e.g., Thimerosal, benzyl alcohol, polyquaternium), antioxidants (e.g., ascorbic acid, sodium metabisulfite), tonicity controlling agents, absorption delaying agents, adjuvants, bulking agents (e.g., lactose, mannitol) and the like. The use of carriers and/or excipients in the field of drugs and supplements is well known. Except for any conventional media or agent that is incompatible with the target health-promoting substance or with the composition, carrier or excipient use in the subject compositions may be contemplated.

In one embodiment, the compositions of the subject invention can be made into aerosol formulations so that, for example, it can be nebulized or inhaled. Suitable pharmaceutical formulations for administration in the form of aerosols or sprays are, for example, powders, particles, solutions, suspensions or emulsions. Formulations for oral or nasal aerosol or inhalation administration may also be formulated with carriers, including, for example, saline, polyethylene glycol or glycols, DPPC, methylcellulose, or in mixture with powdered dispersing agents or fluorocarbons. Aerosol formulations can be placed into pressurized propellants, such as dichlorodifluoromethane, propane, nitrogen, fluorocarbons, and/or other solubilizing or dispersing agents known in the art. Illustratively, delivery may be by use of a single-use delivery device, a mist nebulizer, a breath-activated powder inhaler, an aerosol metered-dose inhaler (MDI), or any other of the numerous nebulizer delivery devices available in the art. Additionally, mist tents or direct administration through endotracheal tubes may also be used.

In one embodiment, the compositions of the subject invention can be formulated for administration via injection, for example, as a solution or suspension. The solution or suspension can comprise suitable non-toxic, parenterally-acceptable diluents or solvents, such as mannitol, 1,3-butanediol, water, Ringer's solution, or isotonic sodium chloride solution, or suitable dispersing or wetting and suspending agents, such as sterile, non-irritant, fixed oils, including synthetic mono- or diglycerides, and fatty acids, including oleic acid. One illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Water or saline solutions and aqueous dextrose and glycerol solutions may be preferably employed as carriers, particularly for injectable solutions. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

In one embodiment, the compositions of the subject invention can be formulated for administration via topical application onto the skin, for example, as topical compositions, which include rinse, spray, or drop, lotion, gel, ointment, cream, foam, powder, solid, sponge, tape, vapor, paste, tincture, or using a transdermal patch. Suitable formulations of topical applications can comprise in addition to any of the pharmaceutically active carriers, for example, emollients such as carnauba wax, cetyl alcohol, cetyl ester wax, emulsifying wax, hydrous lanolin, lanolin, lanolin alcohols, microcrystalline wax, paraffin, petrolatum, polyethylene glycol, stearic acid, stearyl alcohol, white beeswax, or yellow beeswax. Additionally, the compositions may contain humectants such as glycerin, propylene glycol, polyethylene glycol, sorbitol solution, and 1,2,6 hexanetriol or permeation enhancers such as ethanol, isopropyl alcohol, or oleic acid.

### Methods of Using Compounds of the Subject Invention

In certain embodiments, PAPD5 small molecule inhibitors can be administered to a subject. In certain embodiments, PAPD5 small molecule inhibitors can be administered orally, intranasally, intravenously, intraperitoneally, intramuscularly, intrathecally, intracerebroventricularly, subcutaneously, or preferably, intravenously or intranasally, to a subject. In certain embodiments, the administration of PAPD5 small molecule inhibitors can occur before or, preferably, after symptoms of HD develop. In certain embodiments, the subject to which the PAPD5 small molecule inhibitors is administered is an adult; however, juveniles may also benefit from the administration of PAPD5 small molecule inhibitors, including, human subjects less than about 18, about 16, about 14, about 12, about 10, about 8, about 6, about 4, or about 2 years old.

In certain embodiments, the administration of PAPD5 small molecule inhibitors can suppress cell death, TAK1/MKK4/JNK phosphorylation, and caspase 3 cleavage in a dose-dependent manner. In certain embodiment, the administration of PAPD5 small molecule inhibitors can reduce the phosphorylation levels of TAK1, MKK4 and JNK, as well as suppressed caspase 3 cleavage. In certain embodiment, the administration of PAPD5 small molecule inhibitors can rescue cell death. In certain embodiments, the synaptic defects in neurons can be fully rescued upon the administration of PAPD5 small molecule inhibitors, including the restoration of the puncta number of all three synaptic markers: Synapsin I, Bassoon, and Homer1.

### Materials and Methods

### Experimental Model and Subject Details

### Cell Culture and Transfection

SK-N-MC cells (ATCC) were cultured in DMEM (GE Healthcare BioSciences) supplemented with 10% fetal bovine serum and 1% penicillin-streptomycin. Cells were maintained in a 37° C. humidified cell culture incubator supplemented with 5% CO₂. Plasmid DNA and siRNA/synthetic miRNA transfection was performed using Lipofectamine 2000 (Thermo Fisher Scientific) and Lipofectamine RNAiMAX (Thermo Fisher Scientific), respectively.

### Mouse Primary Cortical Neuron Culture and Transfection

E16-embryos of C57BL6J wildtype mice were sacrificed to obtain the mouse primary cortical neurons as previously described (Peng et al., 2021). In brief, cortical lobes were dissected out in ice-cold PBS-glucose. Meninges were removed and cortices were digested in trypsin solution. Primary cortical neurons were cultured on poly-Llysine-coated (0.05 mg/ml) glass coverslips in 24-well plates (50,000 cells per well) and maintained in a 37° C. humidified cell culture incubator supplemented with 5% CO₂. Prior to transfection and drug treatment, neurons were cultured for 5 days. Mature primary neurons were transfected with DNA constructs together with Lipofectamine LTX and Plus Reagent (Thermo Fisher Scientific). Following the manufacturer's protocol, at 4 hours post-transfection culture medium was refreshed and 400 nM of BCH001 was added to the medium. Neurons were further incubated for 72 hours to allow recovery and ectopic expression.

### Drosophila Stocks

Fly lines gmr-Gal4 (Ellis et al., 1993), UAS-DsRed_{CAG0/100} (Li et al., 2008), UAS-Htt exon1Q93 (Steffan et al., 2001), UAS-flMJDQ27/84 (Warrick et al., 2005), UASCTG_{60/480} (Garcia-Lopez et al., 2008) and UAS-EGFP-CGG₉₀ (Jin et al., 2003) were described previously. The UAS-PAPD5 dsRNA line (GD19799, GD41096, and GD45261) was obtained from Vienna Drosophila RNAi Center. All flies were maintained in cornmeal culture medium and genetic crosses were set up in a 21.5° C. incubator (LMS, Sevenoaks, Kent, UK).

### Mouse Strains and Maintenance

Wildtype mouse strain C57BL/6J and HD transgenic mouse strain B6.CBATg(HDexon1)62Gpb/3J (Mangiarini et al., 1996) were obtained from The Jackson Laboratory. The animals maintained and bred in the Laboratory Animal Services Centre of The Chinese University of Hong Kong (CUHK). Housing conditions were kept at temperature of 22±1° C. and humidity of 40-60%, under a 12 h dark/12 h light cycle. Genomic DNA isolated from tail biopsy was used for the genotyping with following primers: forward, 5′-CCGCTCAGGTTCTGCTTTTA-3′ (SEQ ID NO: 1); and reverse, 5′-TGGAAGGACTTGAGGGACTC-3′ (SEQ ID NO: 2). Non-transgenic littermates were used as controls for all the experiments. All animal procedures were approved by the CUHK Animal Experimentation Ethics Committee.

### Method Details

### Plasmid Construction

To generate PAPD5^{+561/+860} wild-type-luciferase and PAPD5^{+561/+860} YY1 mutant luciferase constructs, the PAPD5^{+561/+860} wild-type and PAPD5^{+561/+860} YY1 mutant DNA sequences were synthesized by GenScript and subcloned into pGL4.17[luc2/Neo] firefly luciferase vector (E6721, Promega) using NheI and HindIII. To generate pRI-CMV/GFP-miR-504 and pRI-CMV/GFP-miR-7, a miR-504 mimic sequence, 5′-GACCCTGGTCTGCACTCTATC-3′ (SEQ ID NO: 3), or a miR-7 mimic sequence, 5′-TGGAAGACTAGTGATTTTGTT-3′ (SEQ ID NO: 4), was embedded in an optimized murine miR-155 scaffold sequence: miR-504 forward: 5′-TGCTG GACCCTGGTCTGCACTCTATC GTTTTGGCCACTGACTGACGATAGAGT AGACCAGGGTC-3′ (SEQ ID NO: 5); miR-504 reverse: 5′-CCTGGACCCTGGTCTACTCTATCGTCAGTCAGTGGCCAAAACGATAGAGTGCA GACCAGGGTCC-3′ (SEQ ID NO: 6); miR-7 forward: 5′-TGCTG TGGAAGACTAGTGATTTGTT GTTTTGGCCACTGACTGACAACAAAATCTAGTCTTCCA-3′ (SEQ ID NO: 7) and miR-7 reverse: 5′-CCTGTGGAAGACTAGATTTTGTTGTCAGTCAGTGGCCAAAACAACAAAATCACTA GTCTTCCAC-3′ (SEQ ID NO: 8). Synthesized DNA fragments were then subcloned into pRICMV/GFP-miRNA vector (V6501, Inovogen) at the BbsI restriction site according to the manufacturer's instructions.

### Immunocytochemistry

Neurons were fixed with 4% paraformaldehyde for 10 minutes, followed by permeabilization (0.2% Triton X-100) for another 10 minutes and blocking (1% BSA) for 1 hour at room temperature. Primary antibody incubation was performed in 1% BSA overnight at 4° C. After washing steps, neurons were incubated with secondary antibodies in 1% BSA in dark for 1 hour at room temperature. Samples were counterstained with 1 μg/ml 4,6-diamidino-2-phenylindole (DAPI) solution for nuclei visualization and then washed with PBS, followed by mounting and examined under a confocal microscope (Leica TCS SP8 high speed imaging system). To examine the neurite arborization and quantify the synaptic markers, primary neurons were stained with MAP2 (1:10,000; ab5392, Abcam) and Synapsin I (1:200; ab8, Abcam), Bassoon (1:200; ab110426, Abcam) or Homer1 (1:200; ab97593, Abcam) antibodies. The z stacked maximal projected images were used for statistical analyses using Photoshop 2020 (Adobe Systems). Ten EGFP-positive neurons per replica were used for the statistical analysis.

### Fluorescence In Situ Hybridization (FISH)

Cells were fixed with 4% paraformaldehyde/DEPC-PBS for 15 minutes and permeabilized with 0.2% Triton X-100/DEPC-PBS for 10 minutes at room temperature.

After washing, cells were hybridized with 30 nM denatured TYE563-labeled Affinity Plus DNA probe (5′-TYE563-C+TGC+TGC+TGCTG+CTG+CTG+CT-3′ (SEQ ID NO: 9); IDT; Urbanek and Krzyzosiak, 2016) in hybridization buffer (50% formamide, 10% dextran sulfate, 2× saline-sodium citrate (SSC) and 50 mM sodium phosphate buffer) for 4 hours at 65° C. After hybridization, cells were washed with 2×SSC/0.1% Tween-20 buffer for 3 times at 65° C., followed by further washing with 0.1×SSC buffer for 3 times at 65° C. Cells were then blocked with 1% BSA in DEPC-PBS for 1 hour at room temperature and stained with anti-YY1 (1:200; ab109237, Abcam) overnight at 4° C. After washing, cells were incubated with secondary antibodies in dark for 1 hour at room temperature. Cell nuclei were counterstained with DAPI solution prior to mounting on cover-slips. Images were obtained using a Leica TCS SP8 confocal microscope.

### Total RNA Extraction and RT-PCR

Total cellular RNA was isolated from SK-N-MC cells or fly heads using TRIzol reagent (Thermo Fisher Scientific), followed by reverse transcription using the ImProm-II™ Reverse Transcription System (Promega) according to the manufacturer's instructions.

Primers used were listed in the Table 1.

TABLE 1

| REAGENT or RESOURCE | SOURCE | IDENTIFIER |
|---|---|---|
| Antibodies | | |
| Anti-MAP2 | Abcam | Cat# ab5392 |
| Anti-Synapsin I | Abcam | Cat# ab8 |
| Anti-Bassoon | Abcam | Cat# ab110426 |
| Anti-Homer1 | Abcam | Cat# ab97593 |
| Anti-YY1 | Abcam | Cat# ab109237 |
| Anti-β-tubulin | Abcam | Cat# ab6046 |
| Anti-p-TAK1 | CST | Cat# 4536 |
| Anti-t-TAK1 | CST | Cat# 4505 |
| Anti-p-MKK4 | CST | Cat# 9156 |
| Anti-t-MKK4 | CST | Cat# 9152 |
| Anti-p-MKK7 | CST | Cat# 4171 |
| Anti-t-MKK7 | CST | Cat# 4172 |
| Anti-p-JNK | CST | Cat# 9251 |
| Anti-t-JNK | CST | Cat# 9252 |
| Anti-TAB1 | CST | Cat# 3226 |
| Anti-TAB2 | CST | Cat# 3745 |
| Anti-cleaved caspase 3 | CST | Cat# 9664 |
| Anti-myc | CST | Cat# 2276 |
| Anti-polyQ | Sigma-Aldrich | Cat# P1874 |
| Anti-HA | Sigma-Aldrich | Cat# H3663 |
| Anti-PAPD5 | Invitrogen | Cat# PA5-46747 |
| Anti-GFP | Clontech | Cat# JL-8 |
| Goat anti-rabbit secondary antibody | Jackson | Cat# 11-035- |
| | ImmunoResearch | 045 |
| Goat anti-mouse secondary antibody | Jackson | Cat# 115-035- |
| | ImmunoResearch | 062 |
| Chemicals, peptides, and recombinant | | |
| proteins | | |
| 5Z-7-oxozeaenol (TAK1 inhibitor) | Merck | Cat# O9890 |
| SP600125 (JNK inhibitor) | Merck | Cat# 420119 |
| BCH001 (PAPD5 inhibitor) | Selleck | Cat# S8977 |
| | Chemicals | |
| Critical commercial assays | | |
| miRCURY LNA miRNA miRNome PCR | Qiagen | Cat# 339322 |
| Panels | | |
| Experimental models: Cell lines | | |
| SK-N-MC cells | ATCC | Cat# HTB-10 |
| Experimental models: Organisms/strains | | |
| Mouse: C57BL/6J | Jackson | Strain# 000664 |
| | Laboratory | |
| Mouse: R6/2: B6CBA- | Jackson | Strain# 006494 |
| Tg(HDexon1)62Gpb/3J | Laboratory | |
| D. melanogaster: gmr-Gal4 | Ellis et al., 1993 | N/A |
| D. melanogaster: UAS-DsRed_{CAG0/100} | Li et al., 2008 | N/A |
| D. melanogaster: UAS-Htt exon1Q93 | Steffan et al., | N/A |
| | 2001 | |
| D. melanogaster: UAS-flMJDQ27/84 | Warrick et al., | N/A |
| | 2005 | |
| D. melanogaster: UAS-CTG_{60/480} | Garcia-Lopez et | N/A |
| | al., 2008 | |
| D. melanogaster: UAS-EGFP-CGG₉₀ | Jin et al., 2003 | N/A |
| D. melanogaster: UAS-PAPD5 dsRNA | Vienna | GD19799 |
| | Drosophila | |
| | RNAi Center | |
| Oligonucleotides | | |
| siPAPD5: ON-TARGETplus | Dharmacon | Cat# L- |
| SMARTpool | | 01001100-0005 |
| siYY1: ON-TARGETplus SMARTpool | Dharmacon | Cat# L- |
| | | 01179600-0005 |
| Negative control siRNA | Dharmacon | Cat# D- |
| | | 00121001-50 |
| miRNA: miR-10a-5p: | IDT | N/A |
| 5′- | | |
| UACCCUGUAGAUCCGAAUUUGUG- | | |
| 3′ (SEQ ID NO: 10) | | |
| miRNA: miR-504-5p: | IDT | N/A |
| 5′-AGACCCUGGUCUGCACUCUAUC-3′ | | |
| (SEQ ID NO: 11) | | |
| miRNA: miR-331-3p: | IDT | N/A |
| 5′-GCCCCUGGGCCUAUCCUAGAA-3′ | | |
| (SEQ ID NO: 12) | | |
| miRNA: let-7b-5p: | IDT | N/A |
| 5′-UGAGGUAGUAGGUUGUGUGGUU-3′ | | |
| (SEQ ID NO: 13) | | |
| miRNA: miR-22-5p: | IDT | N/A |
| 5′-AGUUCUUCAGUGGCAAGCUUUA-3′ | | |
| (SEQ ID NO: 14) | | |
| miRNA: miR-7-5p: | IDT | N/A |
| 5′-UGGAAGACUAGUGAUUUUGUUGUU-3′ | | |
| (SEQ ID NO: 15) | | |
| FISH TYE563-labeled Affinity Plus DNA | IDT | N/A |
| probe: 5′- | | |
| TYE563- | | |
| C+TGC+TGC+TGCTG+CTG+CTG+CT-3′ | | |
| (SEQ ID NO: 9) | | |
| Primer: human PAPD5-forward: | Life Technologies | N/A |
| 5′-TGCCCCTAGAGACGACCAA-3′ (SEQ | | |
| ID NO: 16) | | |
| Primer: human PAPD5-reverse: | Life Technologies | N/A |
| 5′-GTAGTTGAGTCCATACGTGCTG-3′ | | |
| (SEQ ID NO: 17) | | |
| Primer: fly PAPD5-forward: | Life Technologies | N/A |
| 5′-TAGACTACTACGGCCGCAAG-3′ | | |
| (SEQ ID NO: 18) | | |
| Primer: fly PAPD5-reverse: | Life Technologies | N/A |
| 5′-AGCTCCTCCCTATGTCGTTG-3′ (SEQ | | |
| ID NO: 19) | | |
| Primer: CAG-forward: | Life Technologies | N/A |
| 5′-AAAAACAGCAGCAAAAGC-3′ (SEQ | | |
| ID NO: 20) | | |
| Primer: CAG-reverse: | Life Technologies | N/A |
| 5′-TCTGTCCTGATAGGTCC-3′ (SEQ ID | | |
| NO: 21) | | |
| Primer: β-actin-forward: | Life Technologies | N/A |
| 5′-ATGTGCAAGGCCGGTTTCGC-3′ (SEQ | | |
| ID NO: 22) | | |
| Primer: β-actin-reverse: | Life Technologies | N/A |
| 5′-CGACACGCAGCTCATTGTAG-3′ (SEQ | | |
| ID NO: 23) | | |
| Recombinant DNA | | |
| pEGFP_{CAG27/78} | Tsoi et al., 2012 | N/A |
| pEGFP-Htt1-550_{CAG23/89} | Peng et al., 2021 | N/A |
| pcDNA3.1(+)-trMJDCAG_{27/78} | Tsoi et al., 2011 | N/A |
| pcDNA3.1(+)-YY1-myc | Chen et al., 2018 | N/A |
| PAPD5^{+561/+860} wild-type-luciferase | This description | N/A |
| construct | | |
| PAPD5^{+561/+860} YY1 mutant-luciferase | This description | N/A |
| construct | | |
| pRI-CMV/GFP-miR-504 | This description | N/A |
| pRI-CMV/GFP-miR-7 | This description | N/A |
| pCMV6-PAPD5-myc-DDK | OriGene Technologies | Cat# RC229323 |
| Software and algorithms | | |
| ImageJ | Schneider et al., 2012 | See worldwide |
| | | website: |
| | | imagej.nih.gov/ij/ |
| GraphPad Prism 8 | GraphPad Software | See worldwide |
| | | website: graph |
| | | pad.com/scientific- |
| | | software/prism/ |
| Photoshop 2020 | Adobe | See worldwide |
| | | website: |
| | | adobe.com/products/ |
| | | photoshop.html |

### Micro RNA Extraction and Real-Time PCR Screening

Micro RNA species from SK-N-MC cells were purified using RNeasy Mini (Qiagen) and RNeasy MinElute Cleanup (Qiagen) kits, followed by reverse transcription using miRCURY® LNA® RT system (Qiagen), according to the manufacturer's instructions. Samples were then subjected to miRCURY LNA miRNA miRNome PCR Panels (Qiagen) for human miRNAs screening under miRCURY® LNA® miRNA real-time PCR (Qiagen) system, following the manufacturer's instructions. Relative miRNA expression was analyzed using the 2^{-ΔΔCT} method.

Functional Analysis of miRNAs Target Genes

Target prediction of miRNAs was performed using miRDB (5.0) (Chen and Wang, 2020) and TargetScan (7.2)(Agarwal et al., 2015). A total of 7,158 target genes were identified by both miRDB and TargetScan. The intersection of target genes predicted by both databases were used for functional analysis using KOBAS (Bu et al., 2021). We focused on the top ten enriched KEGG pathways, which are mainly related to cell survival.

### Protein Sample Preparation, Western Blotting and Antibodies Used

Proteins were extracted from SK-N-MC cells or mouse primary cortical neurons using SDS sample buffer. For protein extraction from mice, half of the brain was homogenized in protein lysis buffer (50 mM Tris-HCl, pH 7.6, 150 mM NaCl, 10% NP40, 5% sodium deoxycholate). All protein samples were boiled at 99° C. for 10 minutes, prior to Western blotting. Primary antibodies used were anti-PAPD5 (PA5-46747, 1:1,000) from Invitrogen; anti-p-TAK1 (4536, 1:1,000), anti-t-TAK1 (4505, 1:2,000), anti-p-MKK4 (9156, 1:1,000), anti-t-MKK4 (9152, 1:2,000), anti-p-MKK7 (4171, 1:1,000), anti-t-MKK7 (4172, 1:2,000), anti-p-JNK (9251, 1:2,000), anti-t-JNK (9252, 1:2,000), anti-TAB1 (3226, 1:2,000), anti-TAB2 (3745, 1:2,000), anti-cleaved caspase 3 (9664, 1:500) and anti-myc (2276, 1:2,000) from Cell Signaling Technology; antipolyQ (P1874, 1:1,000) and anti-HA (H3663, 1:2,000) from Sigma-Aldrich; anti-YY1 (ab109237, 1:2000) and anti-β-tubulin (ab6046, 1:2,000) from Abcam, and anti-GFP (JL-8, 1:4,000) from Clontech. Secondary antibodies used were goat anti-rabbit (11035-045, 1:5,000) and goat anti-mouse (115-035-062, 1:5,000) from Jackson ImmunoResearch.

### Prediction of Transcription Factor Binding Sites on PAPD5 Promoter

The human, rat and mouse PAPD5 promoter sequences were withdrawn from GenBank under the accession numbers NC_000016.10, NC_051354.1 and NC_000074.7, respectively. Transcription factor binding sites were predicted using Transcription factor affinity prediction (see worldwide website: trap.molgen.mpg.de/cgi-bin/trap_form.cgi) (Thomas-Chollier et al., 2011) and PROMO (see worldwide website: alggen.lsi.upc.es/cgibin/promo_v3/promo/promoinit.cgi?dirDB=TF_8.3.) (Messeguer et al., 2002) software.

### Luciferase Assay

The luciferase assay was carried out as described previously (Chen et al., 2018). The pTK-RL Renilla luciferase vector (E2241, Promega) was used as an internal control reporter construct for the normalization of transfection efficiency. Both firefly and Renilla luminescence were recorded on a Spark® multimode microplate reader (Tecan). The relative luciferase activity was calculated by dividing the firefly luminescence reading by the Renilla luminescence reading.

### Lactate Dehydrogenase (LDH) Assay

The LDH assay on SK-N-MC cells were performed according to manufacturer's instructions (CytoTox 96 Non-Radioactive Cytotoxicity Assay Kit, Promega).

### Pseudopupil Assay

12 days-post-eclosion (dpe) adult flies were used for the pseudopupil assay as described previously (Wong et al., 2008). Each fly eye was examined using an Olympus CX31 stereomicroscope. The images of ommatidia were captured using a SPOT Insight CCD camera (Diagnostic instruments Inc., Sterling Heights, MI, USA), and all images were processed using the SPOT Advanced software (Version 5.2; Diagnostic instruments Inc., Sterling Heights, MI, USA) and Photoshop 2020 (Adobe Systems). Two hundred ommatidia collected from 20 individual fly eyes were examined for each condition and used for statistical analysis. The average number of rhabdomeres per ommatidia was presented as the indication of ommatidial integrity.

### External Eye Examination

Adult flies of 12 dpe were anesthetized with C02 and examined using an Olympus SZX12 stereomicroscope. External eye images were captured by a SPOT Insight CCD camera and all the images were processed using the SPOT Advanced software and Adobe Photoshop 2020 software. A total of 10 adult fly eyes from 10 individual flies were examined under the microscope for each condition.

### Quantification and Statistical Analysis

Differences between each group were determined using one-way ANOVA followed by post hoc Tukey's test. NS denotes not significant. *, ** and *** represent P<0.05, P<0.01 and P<0.001, respectively, which are considered statistically significant.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1—Expanded Cag RNA Induces PAPD5 Expression

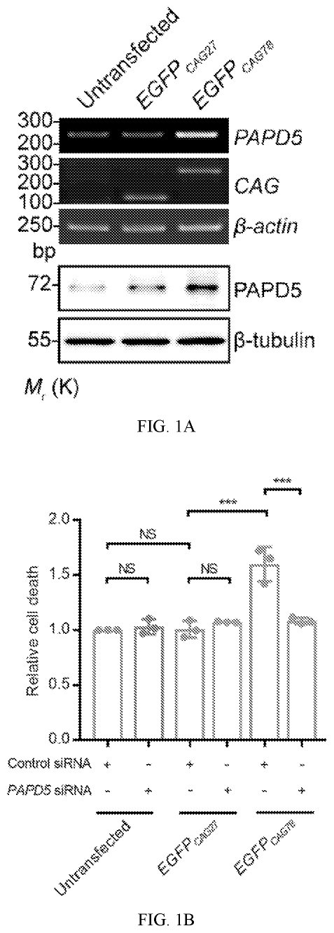

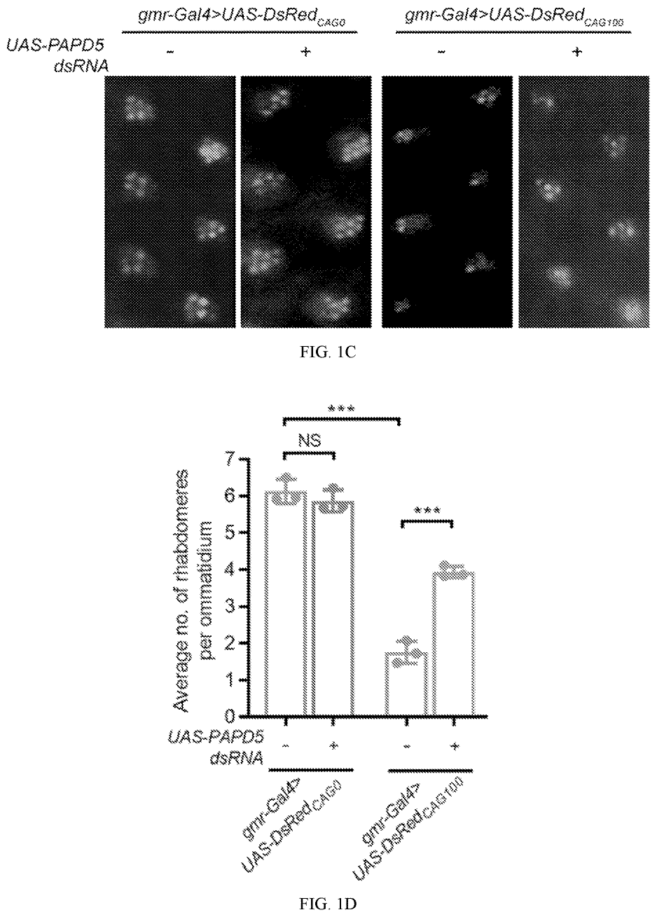

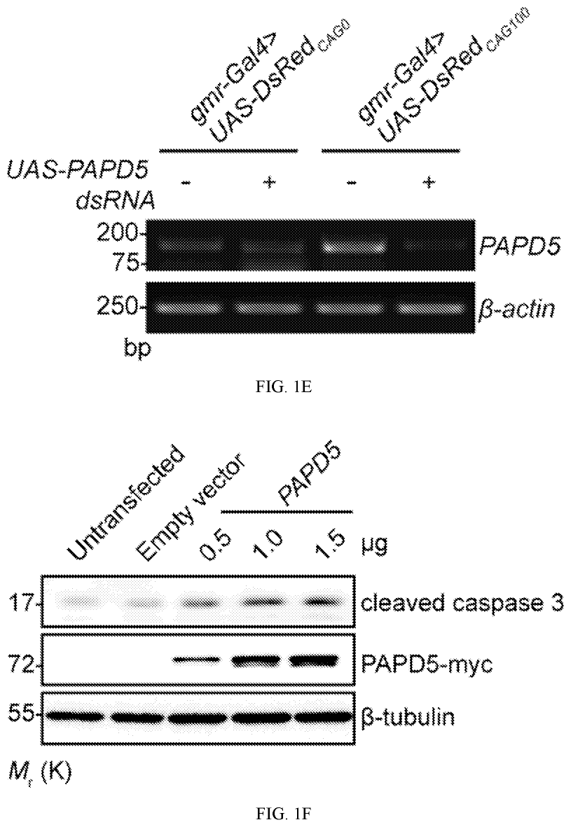

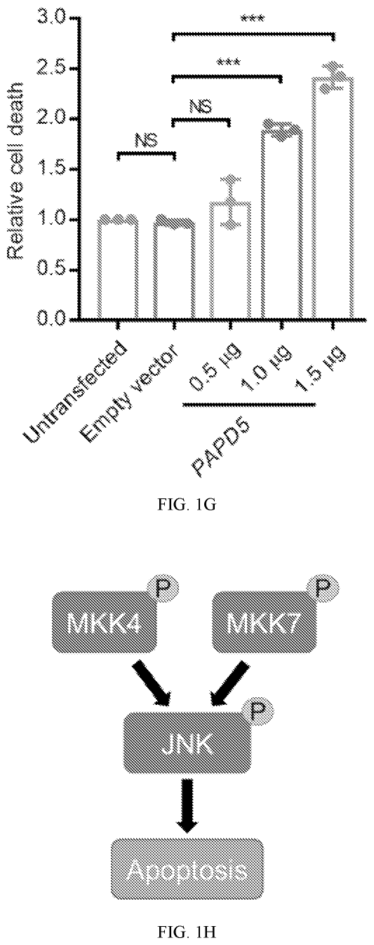

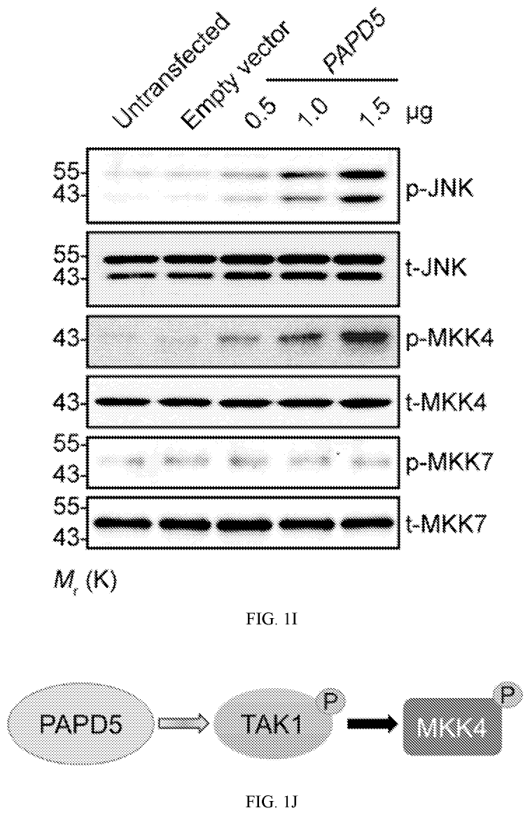

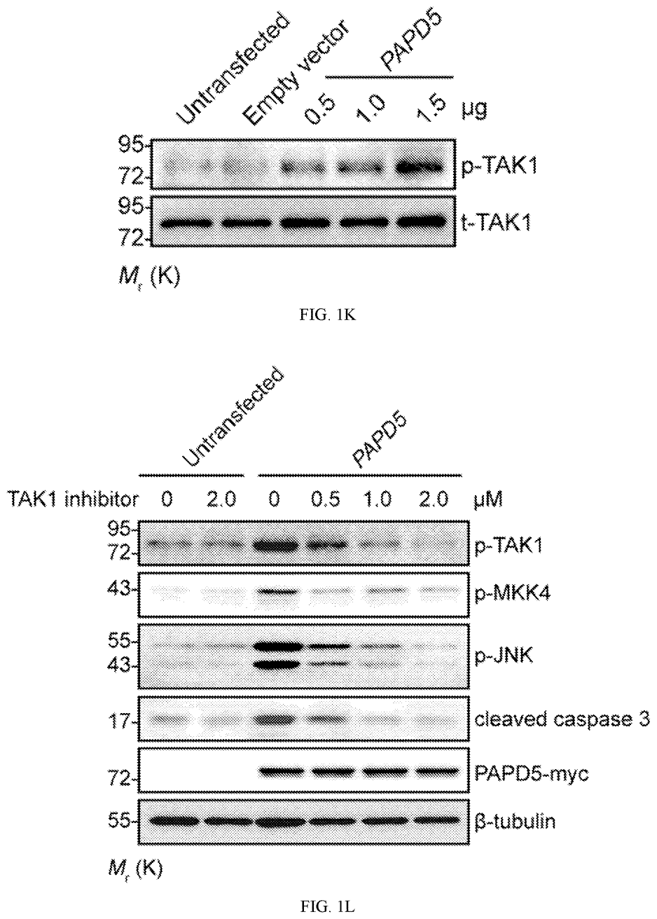

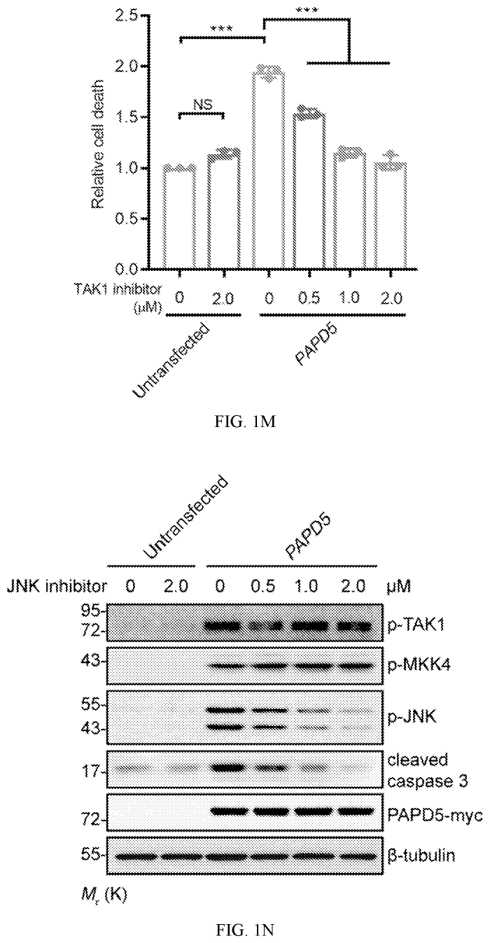

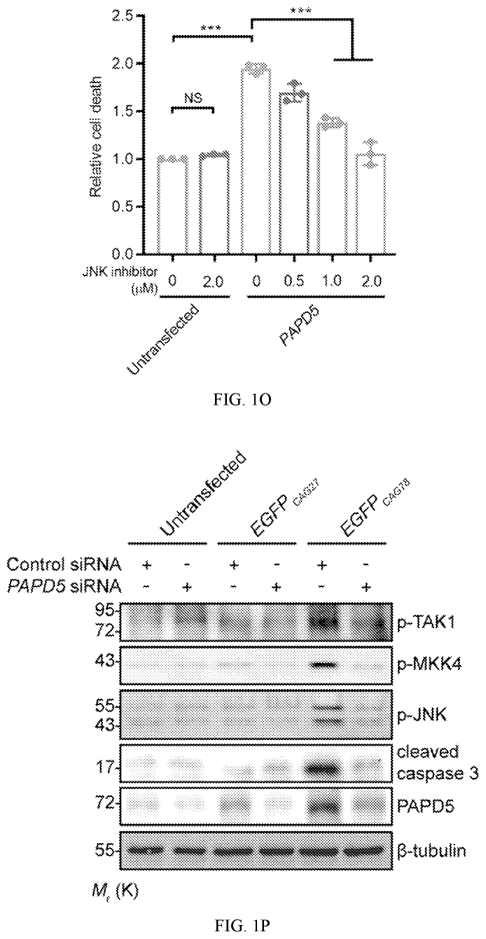

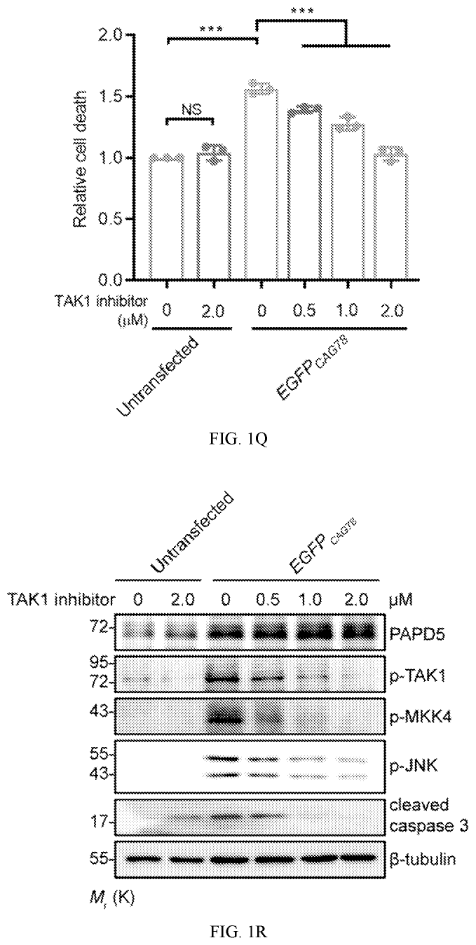

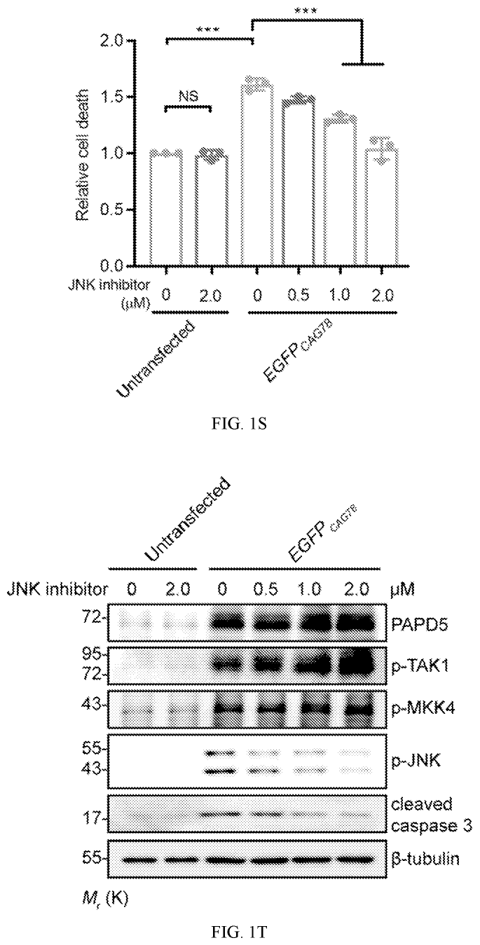

Our recent RNA-seq analysis showed that PAPD5 expression is upregulated in SK-NMC neuroblastoma cells expressing the non-translatable expanded CAG transcripts, EGFP_{CAG78} (Peng et al., 2021; FIG. 1A). This indicates that the mutant expanded

CAG transcript is capable of inducing PAPD5 expression and that an increase in PAPD5 activity may be involved in the RNA toxicity of neurodegeneration in polyQ diseases. We and others have demonstrated that expanded CAG RNA induces cell death (Marti, 2016; Peng et al., 2021; Tsoi et al., 2012). When PAPD5 expression was knocked down in EGFP_{CAG78}-expressing SK-N-MC cells, cell death, as measured by the lactate dehydrogenase (LDH) assay (Peng et al., 2021), was suppressed (FIG. 1B). Our findings showed that PAPD5 plays a crucial role in CAG RNA-induced cytotoxicity. Apart from inducing cell death in a cell model of polyQ diseases, the expression of untranslated CAG sequences also induces neurodegeneration in Drosophila (Li et al., 2008). When the toxic untranslated DsRed_{CAG100} transgene was expressed using a gmr-Gal4 driver, we also detected an increase in PAPD5 expression levels, as well as photoreceptor degeneration (FIGS. 1C-1E). To determine whether the upregulation of PAPD5 expression plays a role in neurodegeneration, we knocked down PAPD5 expression in DsRed_{CAG100} flies and found that retinal degeneration was significantly rescued (FIGS. 1C and 1D). It is noteworthy that the knockdown of endogenous PAPD5 in the unexpanded CAG repeat control did not trigger cell death in vitro (FIG. 1B) nor induce neurodegeneration in vivo (FIGS. 1C and 1D). Further, we found that the knockdown of PAPD5 was not able to rescue the mutant CGG₉₀

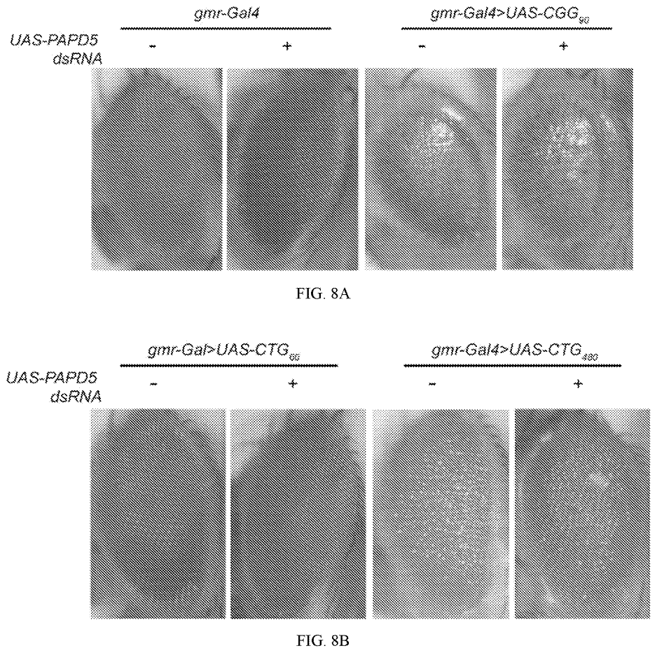

(FIG. 8A) or CUG₄₈₀ (FIG. 8B) RNA-induced external eye phenotypes in Drosophila. Both our in vitro and in vivo observations establish PAPD5 as a specific modifier of expanded CAG RNA toxicity in neurodegeneration in polyQ diseases.

### Example 2—PAPD5 Overexpression Induces Apoptosis Via the TAK1-MKK4-JNK Cascade

We next determined whether the overexpression of PAPD5 is detrimental, and we found that PAPD5 induced caspase 3 activation (FIG. 1F) and cell death (FIG. 1G) in a dose-dependent manner. The mitogen-activated protein kinase (MKK)4/7c-Jun N-terminal kinase (JNK) pathway is a well-established apoptosis-induction mechanism (Wang et al., 2007; FIG. 1H). We detected enhanced phosphorylation levels of JNK and MKK4, but not MKK7, in PAPD5-overexpressing cells (FIG. 1I). Hence, we hypothesized that PAPD5 triggers apoptosis via the MKK4-JNK cascade. Transforming growth factor (TGF)-beta-activated kinase 1 (TAK1) is a MAPK kinase that promotes apoptosis by activating MKK4 and JNK (FIG. 1J; Yang et al., 2004). We further detected prominent phosphorylation of TAK1 in cells overexpressing PAPD5 (FIG. 1K). Remarkably, treating PAPD5-overexpressing cells with a TAK1 kinase inhibitor (5Z-7-oxozeaenol) led to a dose-dependent decrease in TAK1 phosphorylation levels as well as its downstream effectors, including MKK4, JNK and caspase 3 cleavage (FIG. 1L). Consequently, PAPD5-induced cell death was also suppressed by the TAK1 inhibitor in a dose-dependent manner (FIG. 1M). To further delineate the relationship between PAPD5 and the TAK1-MKK4-JNK-caspase cascade, we treated PAPD5-overexpressing cells with a JNK inhibitor (SP600125) and found that both JNK phosphorylation and caspase 3 cleavage were inhibited, while TAK1 and MKK4 phosphorylation levels remained unchanged (FIG. 1N). Similar to TAK1 inhibitor treatment, SP600125 treatment also completely blocked cell death mediated by PAPD5 overexpression (FIG. 1O). In summary, we identified a TAK1-MKK4-JNK pro-apoptotic signaling cascade mediated by PAPD5 overexpression.

We showed that the expression of the mutant expanded CAG transcript in cells induced PAPD5 expression (FIG. 1A) and triggered cell death (FIG. 1B). Upon PAPD5 knockdown, mutant CAG RNA-induced cell death (FIG. 1B) was rescued, which was accompanied by a decrease in the phosphorylation levels of TAK1, MKK4 and JNK, and a suppression of caspase 3 cleavage (FIG. 1P). Consistently, TAK1 and JNK inhibitors rescued mutant CAG transcript-mediated cytotoxicity by specifically targeting TAK1 (FIGS. 1Q and 1R) and JNK (FIGS. 1S and 1T) kinase activities, respectively. Our findings demonstrate a role of PAPD5-mediated TAK1-MKK4-JNK pro-apoptotic pathway activation in the pathogenesis of polyQ diseases.

### Example 3—Cag RNAS Alter the Cellular miRNA Profile Via Upregulating PAPD5 Expression

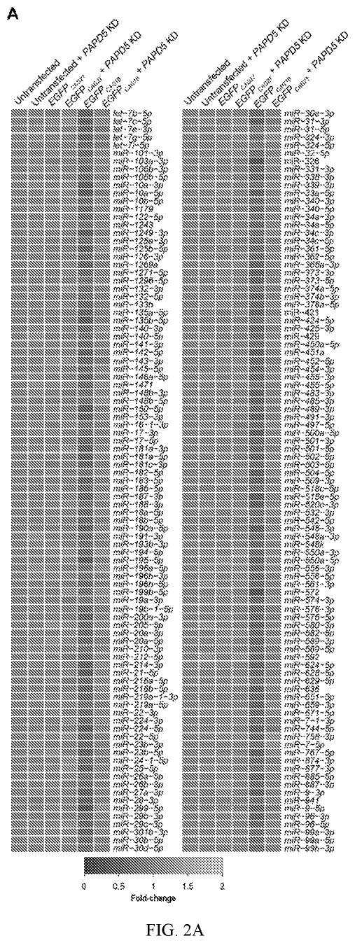

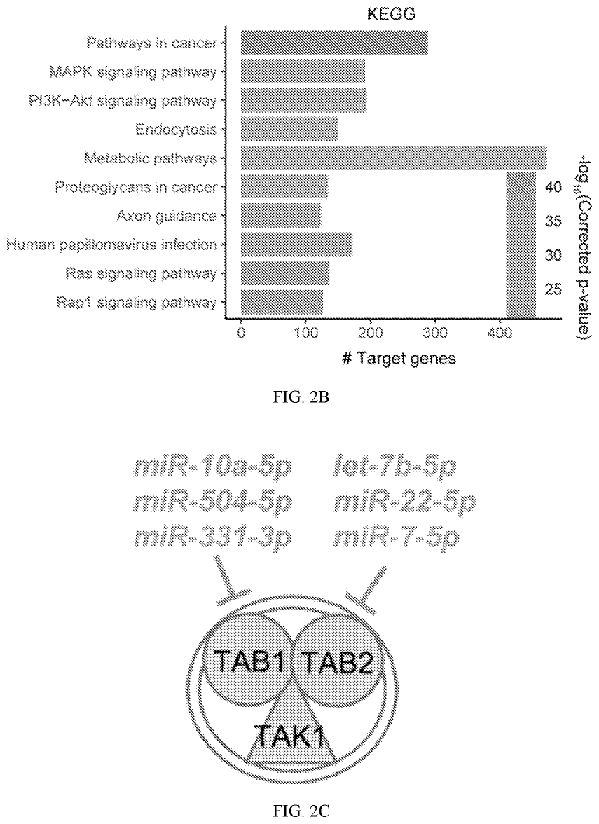

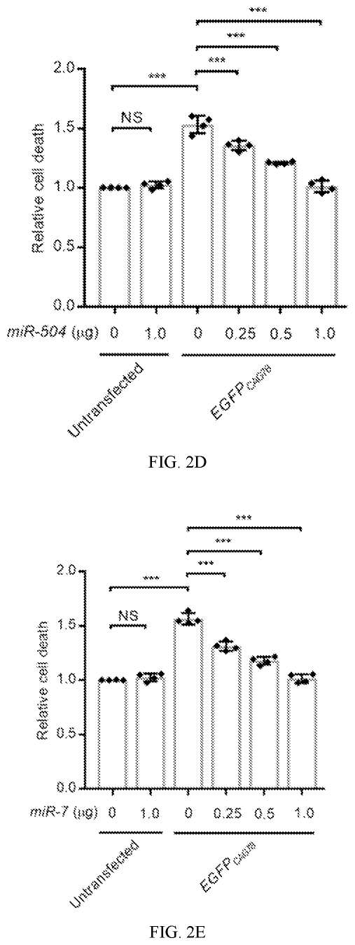

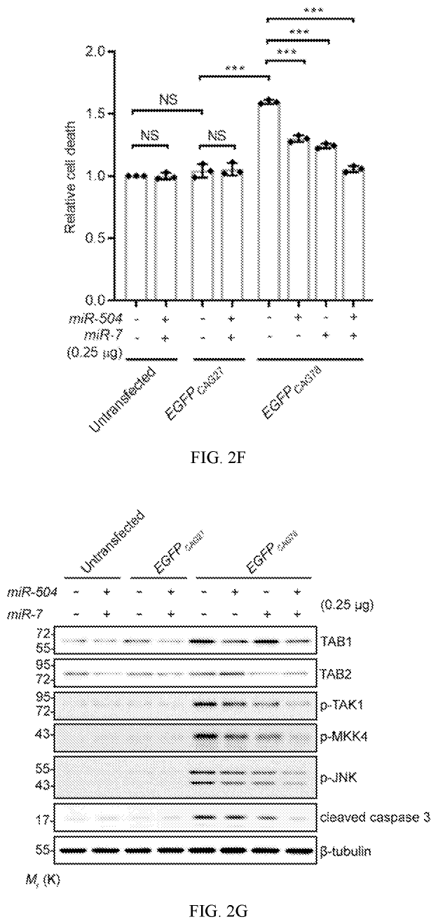

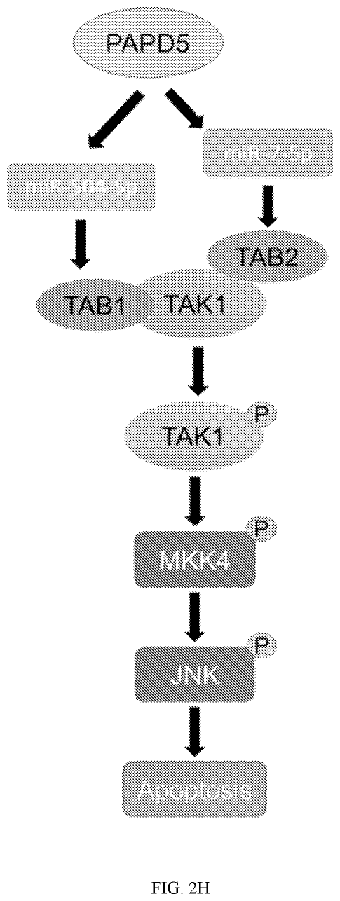

PAPD5 is known to modulate miRNA stability in cells (Boele et al., 2014). We next determined whether PAPD5 upregulation induced by mutant CAG RNA leads to any dysregulation of cellular miRNA profiling. We found that 186 downregulated miRNAs in mutant EGFP_{CAG78}-expressing cells were restored to normal levels upon the knockdown of PAPD5 expression (FIG. 2A). This strongly suggested that PAPD5 plays a pivotal role in the dysregulation of these miRNAs. This subset of PAPD5 regulated miRNAs were predicted to modulate a variety of cellular pathways, including the MAPK signaling pathway (FIG. 2B).

### Example 4—CAG RNAs Downregulate miRNAs that Target Genes Encoding TAK1-Binding Proteins

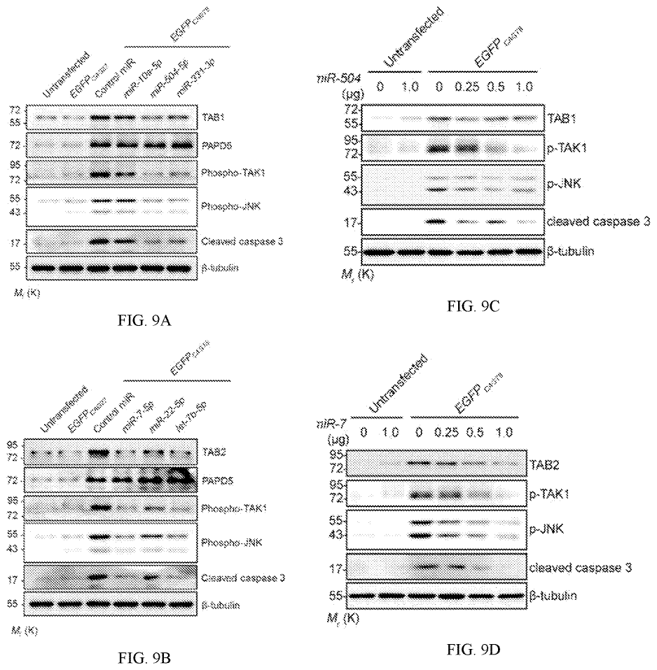

We showed that PAPD5 overexpression induced TAK1, MKK4 and JNK phosphorylation, but did not change the total protein levels of these kinases (FIGS. 1I and 1K). We predicted the targets of the PAPD5-regulated miRNAs (FIG. 2A) and found that miR-10a-5p, miR-504-5p and miR-331-3p targeted TAB1 (Shibuya et al., 1996), while let-7b-5p, miR-22-5p and miR-7-5p targeted TAB2 (Takaesu et al., 2000; FIG. 2C). Both TAB1 and TAB2 are known regulators of TAK1 (Omori et al., 2012; Shibuya et al., 1996; Takaesu et al., 2000), and they play important roles in modulating the activity of TAK1, its downstream MAPK kinase, and cell death pathways (FIGS. 1A-1T; Aashaq et al., 2019). We hypothesized that miRNAs targeting TAB1/TAB2 are degraded by excess PAPD5 protein in mutant CAG RNA-expressing cells, leading to the abnormal upregulation of TAB1/TAB2 expression and the subsequent activation of the TAK1-MKK4-JNK apoptosis signaling cascade. We focused our investigation on miR-504 and miR-7, because they exhibited the most robust gene-silencing effects against TAB1 and TAB2, respectively (FIGS. 9A and 9B). When mutant EGFP_{CAG78} RNA-expressing cells were co-transfected with constructs expressing miRNAs that target either TAB1 (i.e. miR-504; FIG. 2D) or TAB2 (i.e. miR-7; FIG. 2E), cell death was inhibited in a dose-dependent manner. Western blotting analysis further confirmed that the EGFP_{CAG78} RNA-induced phosphorylation of TAK1 and JNK, and cleavage of caspase 3 were suppressed by the overexpression of miR-504 (FIG. 9C) or miR-7 (FIG. 9D). Our data demonstrated that both TAB1 and TAB2 are necessary for mutant CAG transcripts to trigger cell death, because the downregulation of either one of them decreased the ability of TAK1 to induce cell death. When the lowest doses (0.25 μg of miRNA construct; FIGS. 2D and 2E) of miR-504 and miR-7 were used for co-transfection, a prominent suppression of cell death was observed, demonstrating that these miRNAs worked synergistically (FIG. 2F). To explain the associated mechanism, we found that the phosphorylation levels of TAK1, MKK4, JNK, and caspase 3 cleavage were all reduced in our model (FIG. 2G). In summary, we demonstrated that the mutant CAG transcript activates the TAK1-MKK4-JNK caspase cascade via a PAPD5-regulated miRNA pathway (FIG. 2H).

### Example 5—Dysregulation of TAK1-MKK4-JNK Pro-Apoptotic Signaling in HD Models

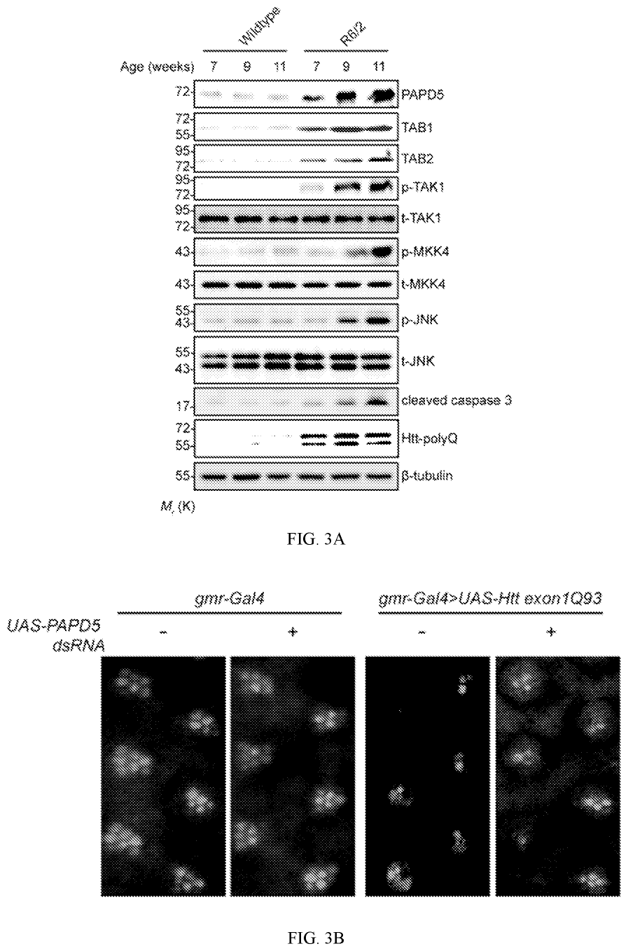

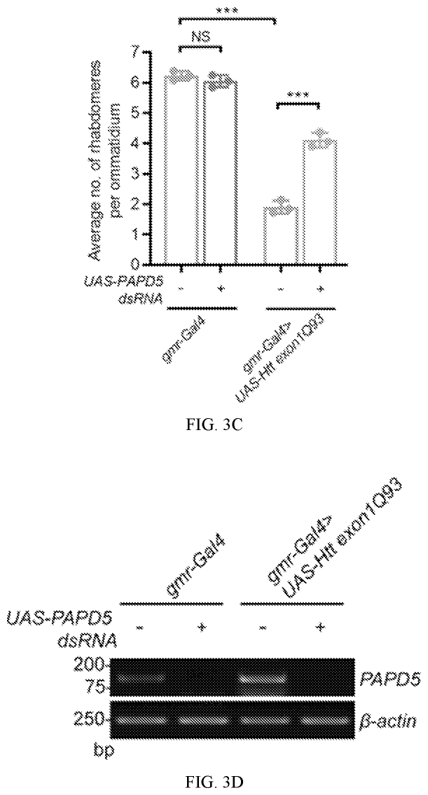

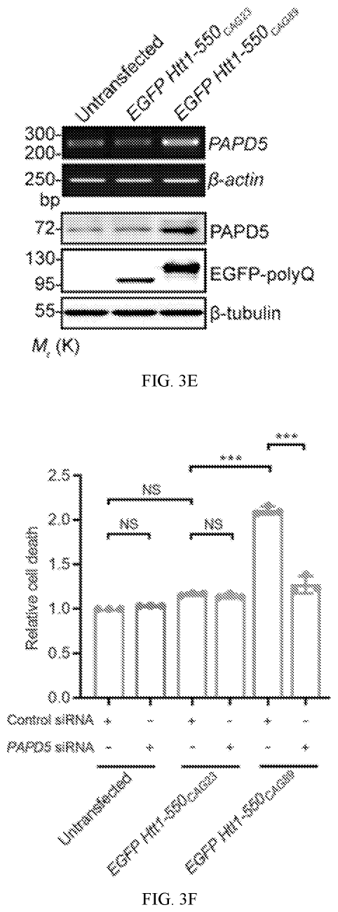

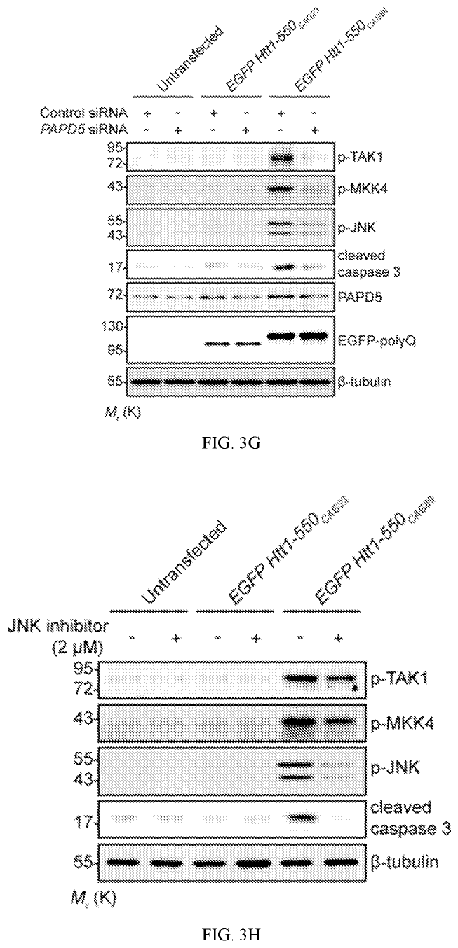

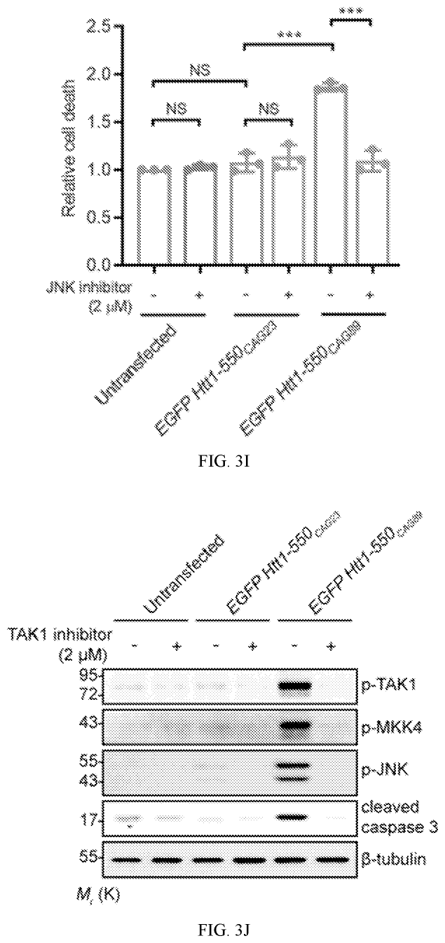

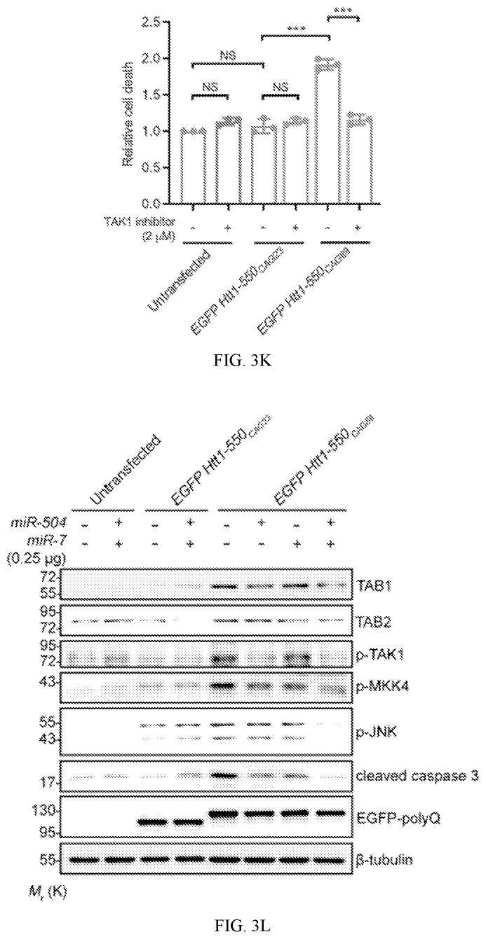

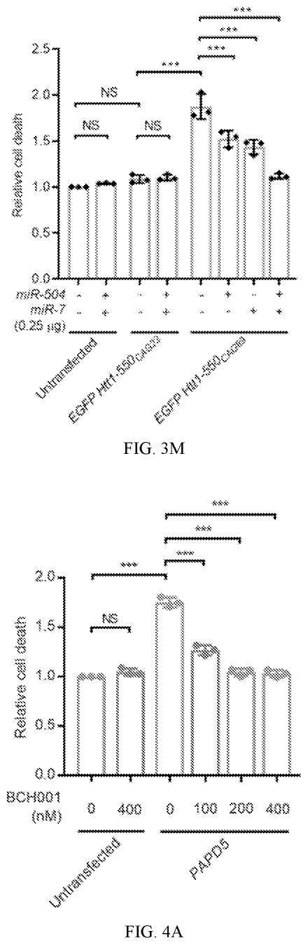

miRNA dysregulation has been reported in HD patients (Johnson et al., 2008; Marti et al., 2010) and in the R6/2 transgenic mouse model of HD (Lee et al., 2011). Further to pure CAG RNA toxicity model (FIGS. 1A-1E), we showed that the expression level of PAPD5 progressively increased in R6/2 HD mouse brains (Mangiarini et al., 1996; FIG. 3A). PAPD5 protein levels correlated well with the phosphorylation status of TAK1, MKK4, JNK, and caspase 3 cleavage (FIG. 3A), and with disease severity under our experimental settings (Peng et al., 2021). When we expressed a mutant UAS-H1t exon1 transgene, Hit exon1Q93 (Steffan et al., 2001), in adult Drosophila using the gmr-GAL4 driver, neurodegeneration was observed in the eyes (FIGS. 3B and 3C). Hit exon1Q93-induced retinal degeneration was suppressed by knocking down PAPD5 expression, as observed by the restoration of rhabdomeric structures in the eyes of adult flies (FIGS. 3B-3D). We then expressed a mutant Hit construct, EGFP-Htt1-550_{CAG89}, in SK-N-MC cells to confirm that PAPD5 mediates the TAK1MKK4-JNK-caspase cascade in HD cell model. Consistent with our EGFP_{CAG78} cell model (FIG. 1A), we detected increased PAPD5 expression at both the transcriptional and translational levels in the EGFP-Htt1-550_{CAG89}-transfected cell model (FIG. 3E). The EGFP-Htt1-550_{CAG89} construct is cytotoxic, but the knockdown of PAPD5 expression rescued most of the EGFP-Htt1-550_{CAG89}-induced cytotoxicity

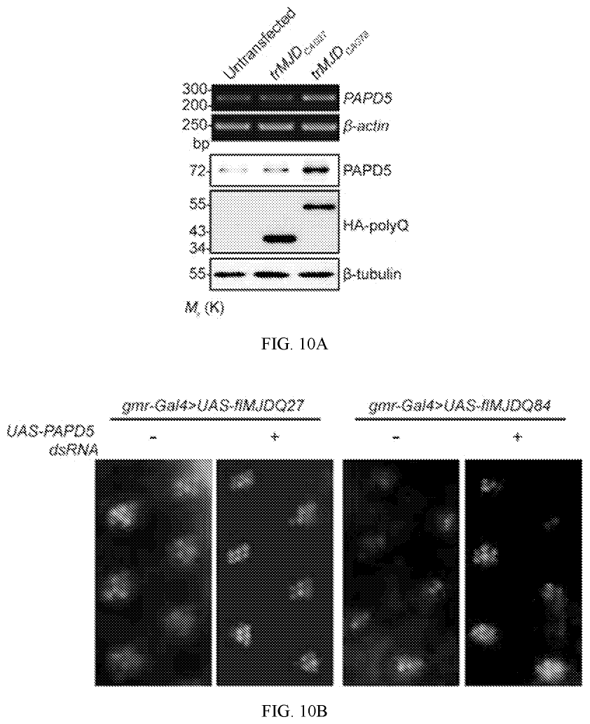

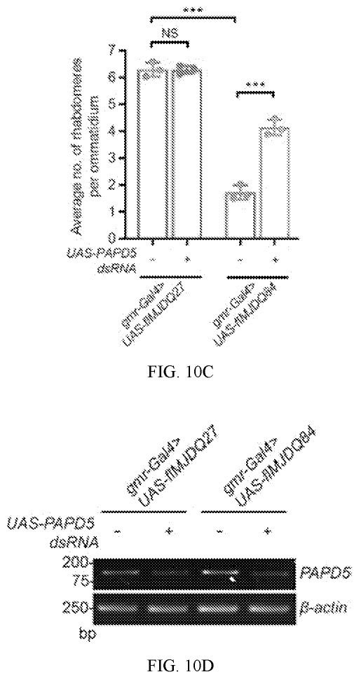

(FIG. 3F). The phosphorylation levels of TAK1, MKK4 and JNK, as well as caspase 3 cleavage were upregulated in EGFP-Htt1-550_{CAG89}-expressing cells compared with cells expressing the unexpanded EGFP-Htt1-550_{CAG23} and untransfected control cells (FIG. 3G). Upon PAPD5 knockdown, both kinase phosphorylation and caspase 3 cleavage were suppressed (FIG. 3G). Treating EGFP-Htt1-550_{CAG89}-expressing cells with JNK inhibitor did not alter TAK1 or MKK4 phosphorylation levels (FIG. 3H), but it suppressed JNK activation, caspase 3 cleavage (FIG. 3H), and cell death (FIG. 3I). The pharmacological inhibition of TAK1 activity in EGFP-Htt1-550_{CAG89} expressing cells diminished TAK1 phosphorylation levels, which subsequently ceased the activation of MKK4 and JNK, and led to the suppression of caspase 3 cleavage (FIG. 3J) and cell death (FIG. 3K). When the expression of TAB1 and TAB2 was knocked down using miR-504 and miR-7, respectively, in EGFP-Htt1-550_{CAG89} expressing cells, TAK1/MKK4/JNK phosphorylation (FIG. 3L), caspase 3 cleavage (FIG. 3L) and cell death (FIG. 3M) were rescued. The suppression of cell death in miR-504/miR-7 co-transfected cells was found to be more prominent and cleaved caspase 3 levels almost returned to baseline levels (FIGS. 3L and 3M). This is in line with previous findings showing that TAB1 and TAB2 double-knockout, but not TAB1 or TAB2 single-knockout, phenocopied TAK1 knockout in mice (Omori et al., 2012). We further observed PAPD5 induction in a cell model of MJD, another polyQ disease (FIG. 10A). The knockdown of PAPD5 expression rescued the retinal degeneration in the eyes of adult MJDQ84 flies (FIGS. 10B-10D). These findings suggested that

PAPD5 may be a general disease modifier of neurodegeneration in polyQ diseases.

### Example 6—Pharmacological Inhibition of PAPD5 Rescues Cell Death and Neuronal Deficits in HD Models

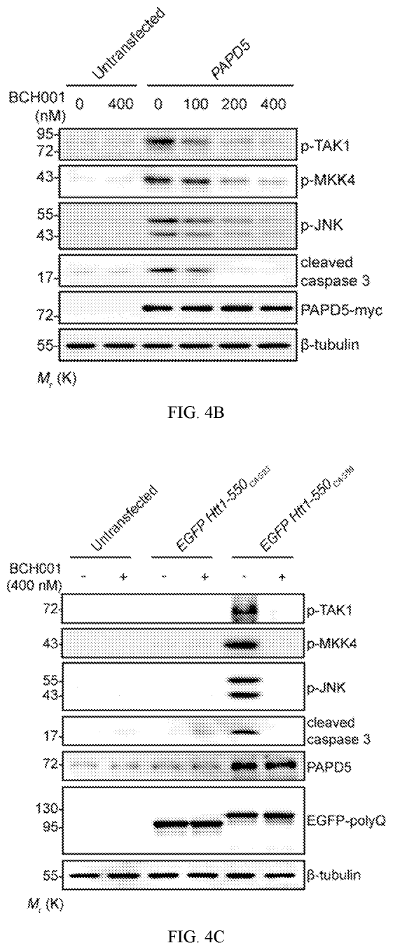

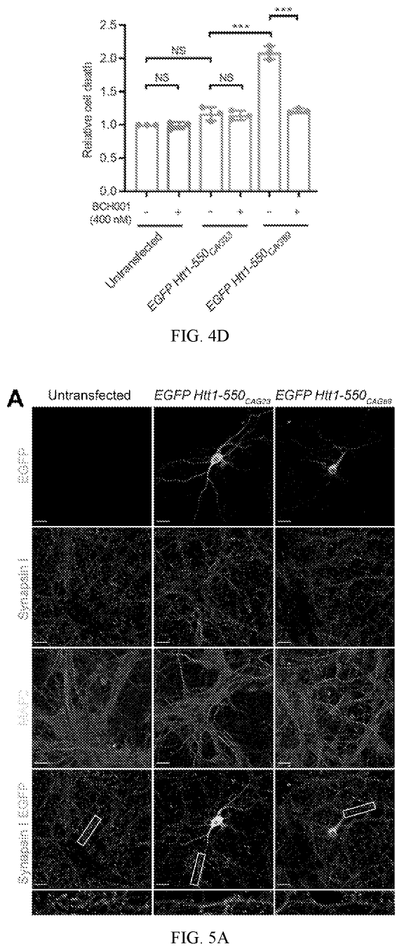

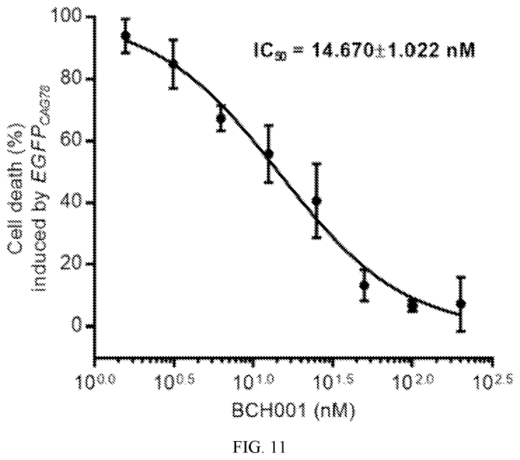

We reported the pro-apoptotic properties of the PAPD5-mediated TAK1-MKK4-JNK caspase cascade (FIGS. 1F-1O), and further demonstrated that PAPD5 expression is increased in cell, Drosophila and mouse models of HD (FIGS. 3A-3M). Thus, inhibiting PAPD5 activity may be an effective therapeutic strategy for HD. BCH001 is a small molecule derived from quinolone. A high-throughput screening study identified it as a PAPD5 inhibitor (Nagpal et al., 2020). We demonstrated that PAPD5 overexpression induced cell death (FIG. 1G). When PAPD5-overexpressing cells were treated with BCH001 at nanomolar concentrations, cell death (FIG. 4A), TAK1/MKK4/JNK phosphorylation, and caspase 3 cleavage (FIG. 4B) were suppressed in a dose-dependent manner. Consistent with the rescuing effect of miRNA overexpression (FIG. 3L), BCH001 treatment reduced the phosphorylation levels of TAK1, MKK4 and JNK, as well as suppressed caspase 3 cleavage in SK-N-MC cells transfected with the mutant EGFP-Htt1-550_{CAG89} construct (FIG. 4C). Cell death triggered by mutant EGFP-Htt1-550_{CAG89} expression was also rescued by BCH001 (FIG. 4D). We further observed that BCH001 treatment completely suppressed mutant EGFP_{CAG78} RNA-induced cell death, with an IC₅₀ value of 14.67 nM (FIG. 11). These findings indicated that the pharmacological inhibition of PAPD5 activity offers therapeutic benefits for HD.

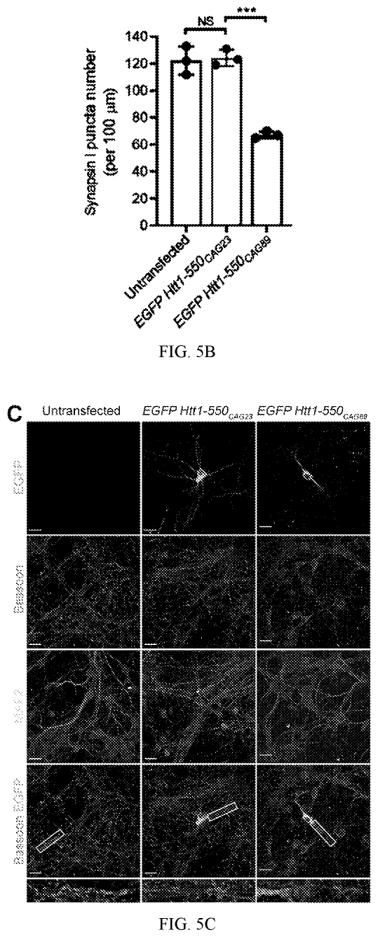

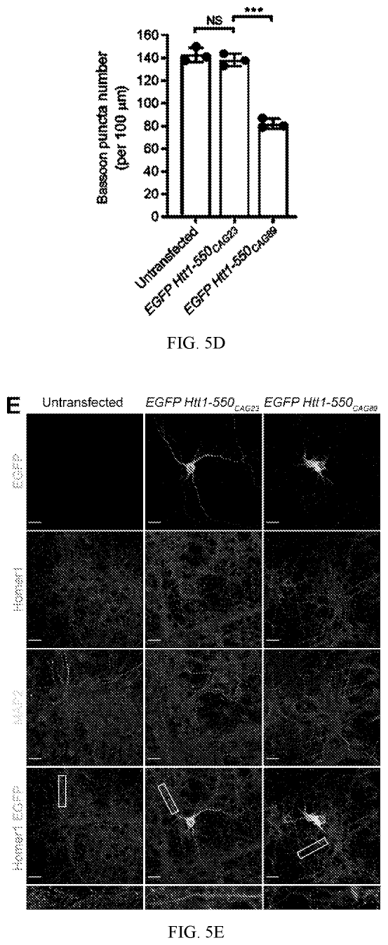

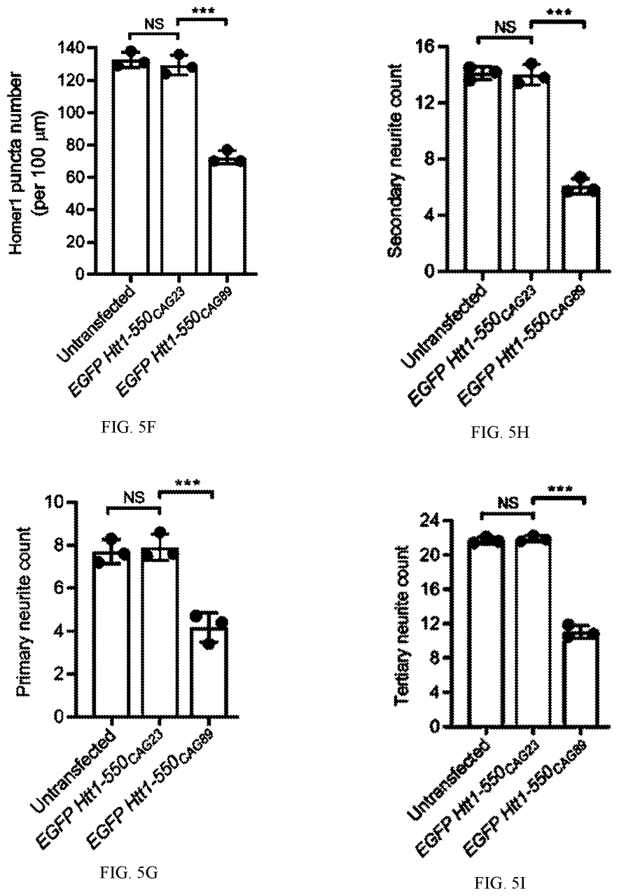

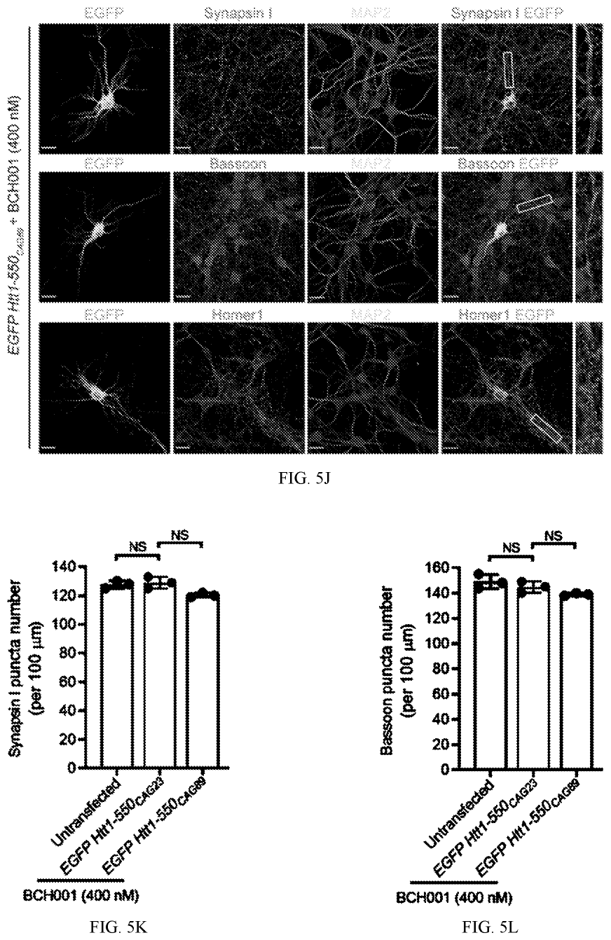

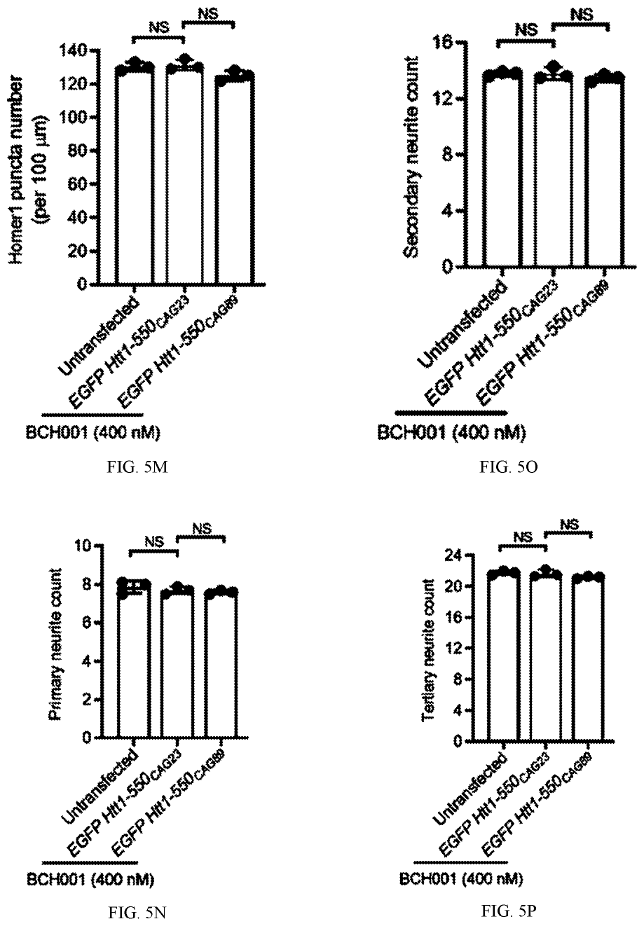

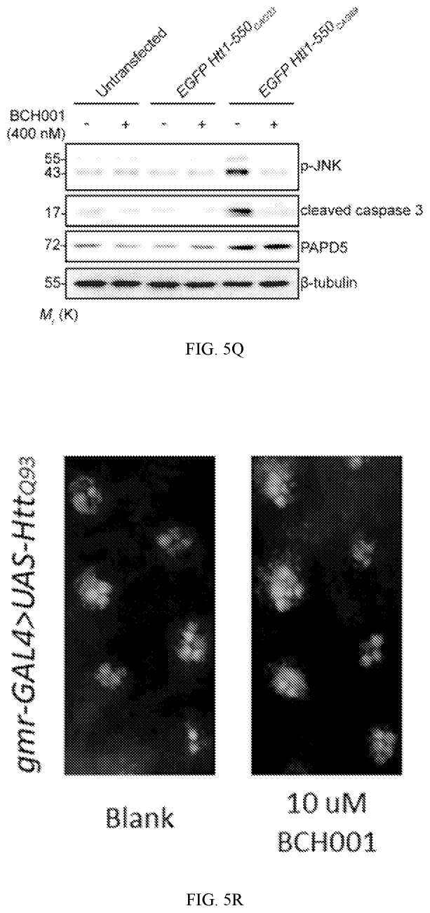

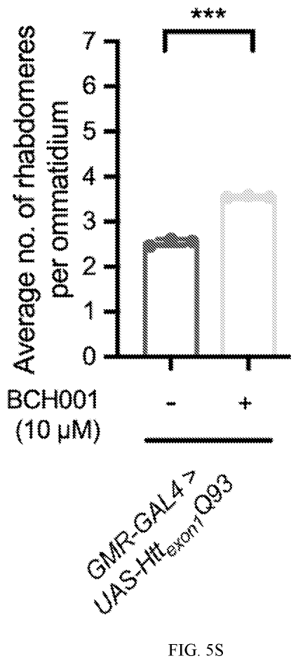

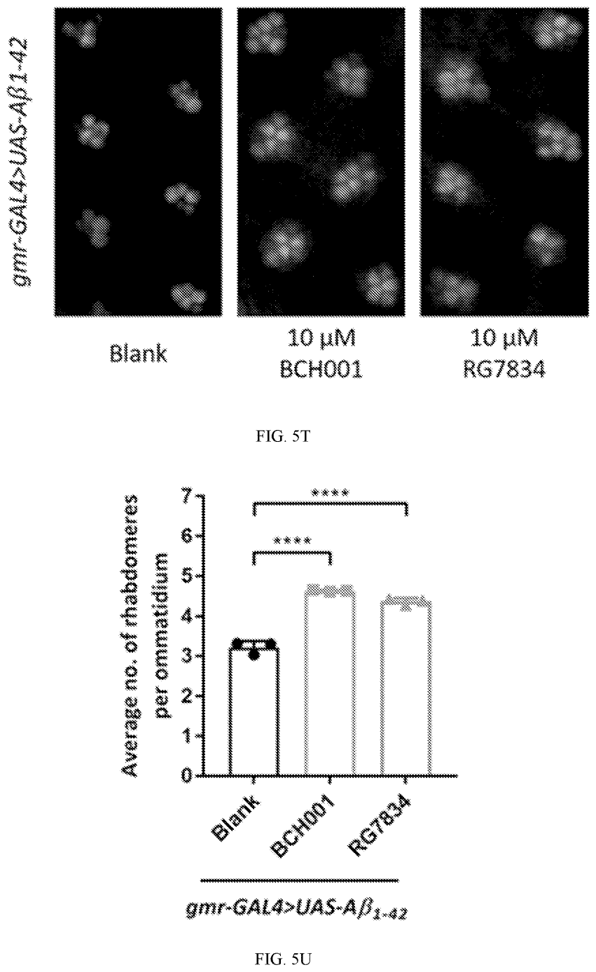

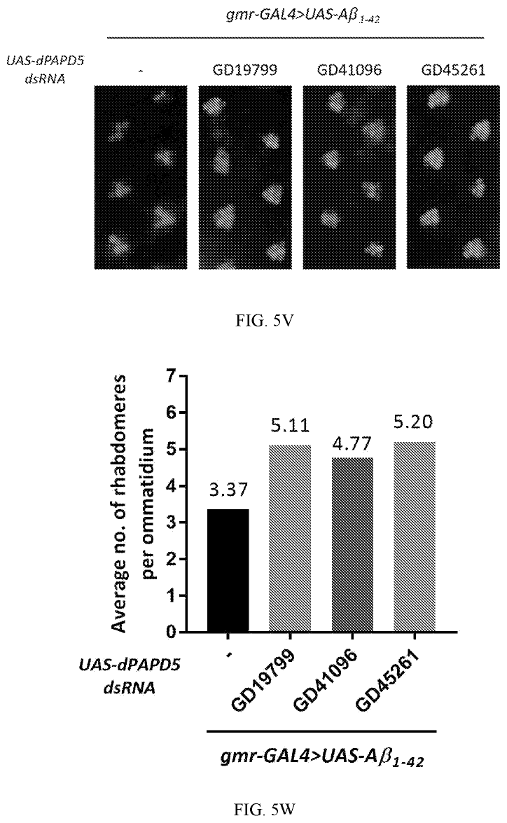

To examine the effect of BCH001 on synaptic defects in neurons, we expressed the mutant EGFP-Htt1-550_{CAG89} construct in mouse primary cortical neurons. Compared with untransfected neurons and neurons that were transfected with the unexpanded EGFP-Htt1-550_{CAG23} construct, neurons expressing EGFP-Htt1-550_{CAG89} displayed fewer puncta of the neuronal synaptic trafficking marker, Synapsin I (FIGS. 5A and 5B), and the pre- and post-synaptic components, Bassoon (FIGS. 5C and 5D) and Homer1 (FIGS. 5E and 5F). This finding indicated that EGFP-Htt1-550_{CAG89} exerts toxic effects on synaptic activities at both the pre- and post-synaptic compartments. We also examined the neurite morphology of these neurons, and found that the expression of EGFP-Htt1-550_{CAG89} caused extensive neurite loss, including the loss of all primary (FIG. 5G), secondary (FIG. 5B) and tertiary neurites (FIG. 5I). More importantly, the synaptic defects in EGFP-Htt1-550_{CAG89}-expressing neurons were fully rescued upon BCH001 treatment, including the restoration of the puncta number of all three synaptic markers: Synapsin I (FIGS. 5J and 5K), Bassoon (FIGS. 5J and 5L) and Homer1 (FIGS. 5J and 5M). Further, all neurites were recovered to their control levels upon BCH001 treatment, as detected in the unexpanded EGFP-Htt1-550_{CAG23} and untransfected controls (FIGS. 5N-5P). At the biochemical level, both JNK phosphorylation and caspase 3 cleavage were attenuated in BCH001-treated EGFPHtt1-550_{CAG89}-expressing neurons (FIG. 5Q). These findings substantiate the suppression effect of BCH001 against PAPD5-induced pro-apoptotic pathway activation in mutant Hit-expressing neurons. When we expressed Htt_{Q93} mutant transgene with the gmr-GAL4 retinal transgene driver in Drosophila, we detected neurodegeneration as indicated by a reduction in rhabdomere numbers (FIGS. 5R and 5S). Rhabdomeres are photo-sensing organelles in photoreceptor neurons. Upon 10 uM BCH001 treatment, a marked suppression of Htt_{Q93}-induced retinal neurodegeneration. We further showed that BCH001 and another PAPD5 inhibitor RG7834 are also capable of suppressing A-beta peptide toxicity in Drosophila (FIGS. 5T and 5U). Based on the PAPD5 inhibition machinery, we further tested if the knockdown of the PAPD5 gene could also rescue A-beta peptide-induced retinal degeneration in Drosophila (FIGS. 5V and 5W). These data suggest PAPD5 inhibition, including PAPD5 inhibitor treatment or PAPD5 gene knockdown or silencing using small interfering RNA (siRNA) or antisense oligonucleotide (ASO) as gene therapy, can be a potential direction of therapeutic development for Alzheimer's Disease.

### Example 7—Yin Yang 1, a Transcriptional Regulator of PAPD5 Expression, is Recruited to RNA Foci and Protein Aggregates in HD Models

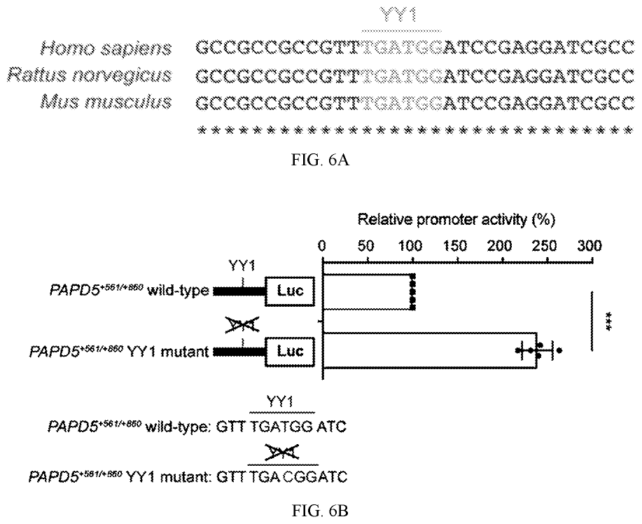

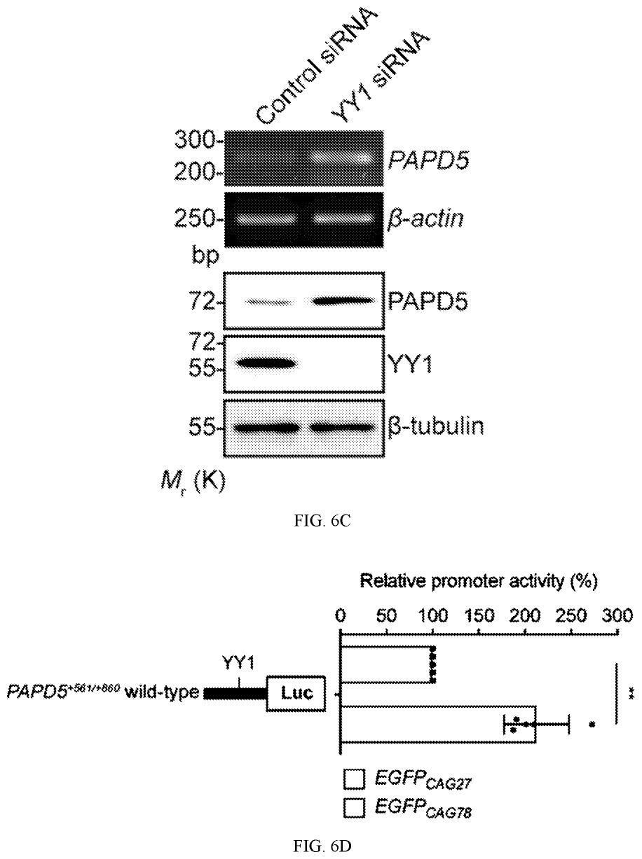

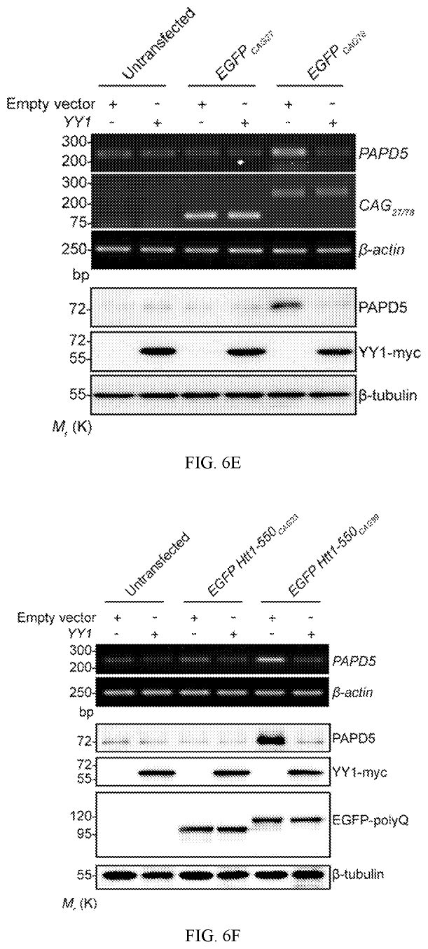

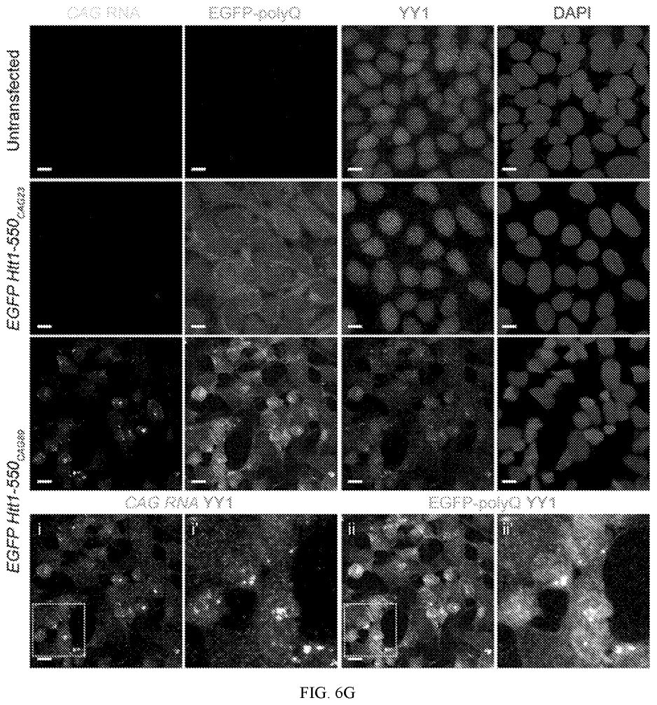

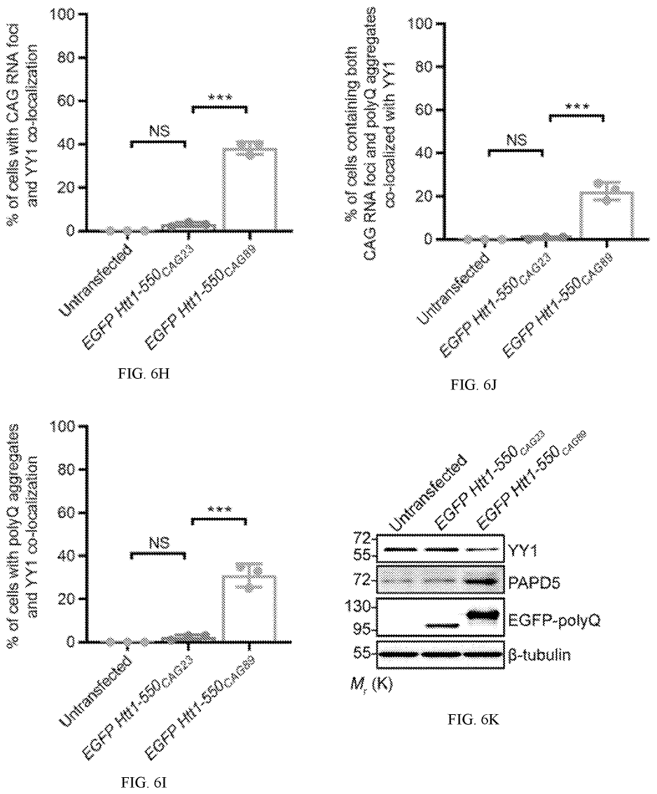

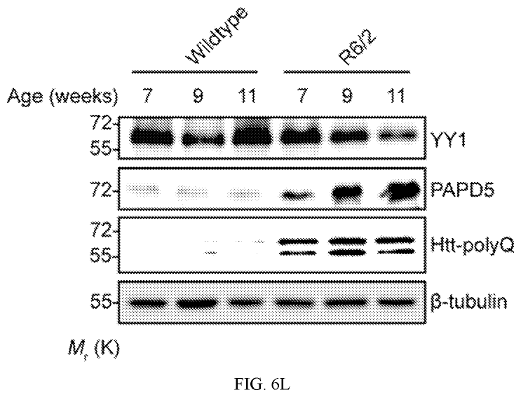

We found that both the transcript and protein levels of PAPD5 were increased in cell, Drosophila and mouse models of HD (FIGS. 3A-3M). This suggested that PAPD5 transcription is dysregulated in HD. We next studied the mechanism through which mutant Htt induces PAPD5 transcription. A putative YY1 transcriptional regulator binding site (TGATGG) was identified within the PAPD5^{+561/+860} promoter region, and this site was found to be highly conserved in mammals (FIG. 6A). The YY1 protein (Shi et al., 1991) is reported to function as a transcriptional repressor to regulate neuronal function (He and Casaccia-Bonnefil, 2008), and we have previously shown that YY1 is involved in polyQ-type spinocerebellar ataxia (Chen et al., 2018). We constructed wild-type and mutant PAPD5^{+561/+860} promoter reporters and performed luciferase assays to examine the role of this YY1 binding site in controlling PAPD5 promoter activity (FIG. 6B). Our data showed that mutating one conserved nucleotide (T>C) in the YY1 binding site resulted in a ˜2.4-fold increase in luciferase activity compared with the activity of the wild-type luciferase construct (FIG. 6B). In addition, we found that YY1 knockdown led to an increase in endogenous PAPD5 expression levels (FIG. 6C). These results indicated that YY1 is a transcriptional repressor of PAPD5.

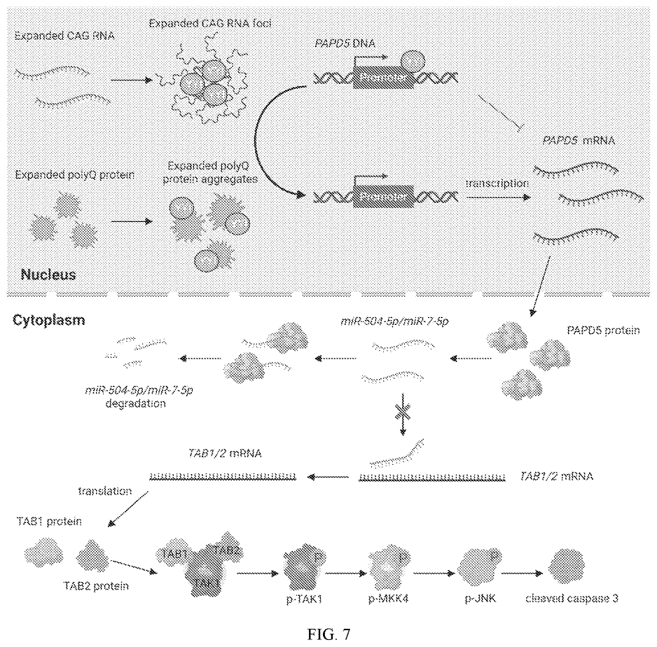

When we measured the luciferase reporter activity of the wild-type PAPD5^{+561/+860} promoter in cells expressing EGFP_{CAG78}, a ˜2.1-fold increase in luciferase activity was detected (FIG. 6D), compared with the activity in cells expressing the unexpanded control, EGFP_{CAG27}. This effect was reminiscent of the response of the luciferase reporter activity of the mutant PAPD5+561/+860 promoter (FIG. 6B). These results indicated that YY1 is involved in the transcriptional dysregulation of PAPD5 in mutant CAG-expressing cells. We next aimed to determine whether YY1 overexpression restored PAPD5 expression in cells transfected with mutant EGFP_{CAG78} (FIG. 6E) and EGFP-Htt1-550_{CAG89} (FIG. 6F) constructs. We found that YY1 overexpression decreased PAPD5 expression at both the RNA and protein levels in mutant-expressing cells compared with cells transfected with the empty vector control. These findings suggested that the transcriptional dysregulation of PAPD5 may be due to an insufficient quantity of functional YY1 protein in mutant cells. In polyQ diseases, endogenous transcriptional regulators are often reported to be recruited to protein aggregates (McCampbell et al., 2000). We next performed in situ hybridization and fluorescence microscopy to examine microscopic CAG RNA foci (in yellow) and polyQ protein aggregates (in green) in EGFP-Htt1-550_{CAG89}-transfected cells (FIG. 6G). In contrast to the untransfected and unexpanded EGFP-Htt1-550_{CAG23} control cells, RNA foci and protein aggregates were detected in the EGFP-Htt1-550_{CAG89}-transfected cells (FIG. 6G). Importantly, we observed that YY1 (in red) was recruited to both CAG RNA foci and polyQ protein aggregates (FIG. 6G). Approximately 38% of the EGFPHtt1-550_{CAG89}-transfected cells showed co-localization of YY1 and the CAG RNA foci (FIG. 6H), while the co-localization of YY1 and polyQ aggregates was found in ˜31% of cells (FIG. 6I). Furthermore, approximately 22% of EGFP-Htt-550_{CAG89} transfected cells displayed the co-localization of YY1 with both CAG RNA foci and polyQ aggregates (FIG. 6J). We further found that the soluble level of YY1 protein decreased in EGFP-Htt1-550_{CAG89}-expressing cells compared with control cells (FIG. 6K). Consistently, a reduction in soluble YY1 protein levels was observed in R6/2 HD mouse brains compared with normal mouse brains (FIG. 6L). These data support the notion that functional YY1 protein is depleted in mutant Htt-expressing cells and animal disease models through its recruitment to both RNA foci and protein aggregates. Consequent to YY1 protein sequestration, PAPD5 transcription was de-repressed, leading to the activation of the TAK1-MKK4-JNK pro-apoptotic pathway in our HD models (FIG. 7).

### Example 8—The Non-Canonical Poly(A) Polymerase PAPD5 is Involved in miRNA Dysregulation in PolyQ Diseases

miRNAs are small regulatory RNAs that are expressed in most tissues, including the brain. They control gene expression by silencing their mRNA targets via the RISC mechanism (Dong and Cong, 2019). The dysregulation of miRNAs has been described in affected brain regions of HD (Marti et al., 2010), indicating that alterations in the mRNA levels of miRNA targets may be involved in disease pathogenesis. TAK1-MKK4-JNK pro-apoptotic pathway was activated in HD models as a consequence of the downregulation of miRNAs, including miR-504-5p and miR-7-5p (FIG. 7).

Most miRNAs are stable in cells because they associate with argonaute proteins to form the RISC complex, which protects them from exonuclease degradation. In neurons, certain miRNAs are found to be more rapidly turned over and this phenomenon may be linked to neuronal activities (Krol et al., 2010). When a miRNA displays extensive complementarity to its mRNA target, it triggers 3′ tailing, followed by miRNA degradation via 3′-5′ trimming. This phenomenon is known as target directed miRNA degradation (TDMD) (Ameres et al., 2010). PAPD5 is one of the enzymes involved in TDMD. This non-canonical poly(A) polymerase catalyzes the posttranscriptional adenylation of miRNAs and triggers their degradation (Boele et al., 2014). We showed that PAPD5 transcript and protein levels were increased in polyQ disease models (FIGS. 1A-1T and 10A-10D), including HD (FIGS. 3A-3M and 5A-5Q). To determine the functional consequence of PAPD5 upregulation, we identified a subset of miRNAs with downregulated expression levels in our model. To determine whether these miRNAs are regulated by PAPD5, we knocked down PAPD5 expression in our cell model and found that the levels of these downregulated miRNAs were restored to control levels (FIG. 2A). These miRNAs are believed to be regulated by PAPD5 via the TDMD degradation pathway (Boele et al., 2014). Boele et al. (2014) demonstrated a positive correlation between PAPD5 expression levels and miRNA adenylation ratio, and showed that a PAPD5-mediated TDMD mechanism exists in different pathological conditions, including cancer and noncancerous proliferative diseases. Both protein aggregates and RNA foci contribute to PAPD5 transcriptional dysregulation in our polyQ disease models. Abnormal protein aggregates, such as mutant polyQ proteins, are known to sequester essential cellular proteins (Lieberman et al., 2019), which removes proteins from their normal subcellular locations, causing neuronal dysfunction. CREB-binding protein is a well-characterized transcriptional regulator with compromised activity in polyQ diseases due to its recruitment to protein aggregates (McCampbell et al., 2000). YY1 functions as a transcriptional repressor (Shi et al., 1991) to regulate gene expression in the nervous system (He and Casaccia-Bonnefil, 2008). We previously reported that YY1 is recruited to polyQ protein aggregates, resulting in transcriptional dysregulation during neurodegeneration in polyQ diseases (Chen et al., 2018). In addition to protein aggregates, repeat expansion RNAs are also reported to recruit cellular proteins (Zhang and Ashizawa, 2017). YY1 is recruited to both CAG RNA foci and polyQ protein aggregates. The depletion of functional YY1 in disease cells led to the de-repression of PAPD5 transcription, resulting in miRNA dysregulation (FIG. 7).

### Example 9—the TAK1-MKK4-JNK Pro-Apoptotic Signaling Pathway in the Pathogenesis of POLYQ Diseases

TAK1 signaling is known to trigger apoptosis (Aashaq et al., 2019; Omori et al., 2006). The inhibition of TAK1 activity conferred neuroprotection in a rodent model of traumatic brain injury (Zhang et al., 2013). The kinase activity of TAK1 is regulated by TABs (Omori et al., 2012). Both TAB1 (Shibuya et al., 1996) and TAB2 (Takaesu et al., 2000) are known activators of TAK1. Tab1/Tab2 double-knockout mice display the same defects in the intestinal epithelium as Tak1 single-knockout mice (Omori et al., 2012). The upregulation of PAPD5 in our HD models causes the increase the expression of both TAB1 and TAB2. We further identified six PAPD5 regulated miRNAs that target TAB1 and TAB2 (FIGS. 1A-1T and 2A-2H). Consistent with our findings, increase of TAB2 expression was reported in caudate nucleus of HD patients (Durrenberger et al., 2012). The dysregulation of TAB1/TAB2 expression is expected to affect TAK1 phosphorylation and trigger a downstream pro-apoptotic pathway. In addition to miRNAs that target the TAB1 and TAB2 genes, we also found that miR143-3p levels were downregulated in our cell model and they were restored upon PAPD5 knockdown (FIG. 2A). miR-143-3p targets TAK1 (Shi et al., 2018; Tu et al., 2020), which further highlights the complexity of TAK1 activity regulation in HD and other polyQ diseases. It has also been reported that the downregulation of miR-1433p, which directly targets TAK1, may promote TAK1 phosphorylation in the liver (Tu et al., 2020). However, miR-143-3p did not affect the total protein levels of TAK1 (FIGS. 1K and 3A). In ovarian cancer, miR-143-3p has been found to downregulate TAK protein levels (Shi et al., 2018). The mechanism whereby miR-1433p affects TAK1 function remains to be elucidated. TGF-beta receptor associates with TAK1 and TNF receptor-associated factor 6 (TRAF6). Ligand-bound TGF-beta receptor induces TRAF6 K-63 poly-ubiquitination, and this in turn activates TAK1 function (Zhang, 2017). TRAF6 protein was found to accumulate in the insoluble fraction of the post-mortem brains of patients with HD (Zucchelli et al., 2011). It is possible that TRAF6 also plays a role in regulating TAK1, thus contributing to the activation of TAK1 in polyQ diseases.

### Example 10—BCH001 as a Modulator of TDMD and a Drug Candidate for POLYQ Toxicity

As a non-canonical poly(A) polymerase, PAPD5 possess enzymatic activity (Boele et al., 2014). In general, enzymes are considered to be desirable drug targets. The small molecule, BCH001, was recently reported to specifically target PAPD5 enzyme activity (Nagpal et al., 2020). Upon treatment with BCH001, the 3′ adenylation of the noncoding RNA template telomerase RNA component (TERC) was reduced. This compound does not induce cell death at 400 nM (FIG. 4A). Treatment with 200 nM BCH001 was sufficient to suppress PAPD5-induced cell death (FIG. 4A). This finding highlights the use of BCH001 as an inhibitor of PAPD5-TDMD.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### EXEMPLIFIED EMBODIMENTS

Embodiment 1. A method of reducing cell death, reducing phosphorylation levels of TAK1, MKK4, or JNK, suppressing caspase 3 cleavage, or rescuing a synaptic defect in a subject, or any combination thereof, comprising:
administering an effective amount of a composition comprising a Poly(A) RNA polymerase D5 (PAPD5) small molecule inhibitor to the subject.

Embodiment 2. The method of embodiment 1, wherein the PAPD5 small molecule inhibitor is derived from quinolone.

Embodiment 3. The method of embodiment 1, wherein the PAPD5 small molecule inhibitor is BCH001, according to formula (I) or RG7834, according to formula (II):

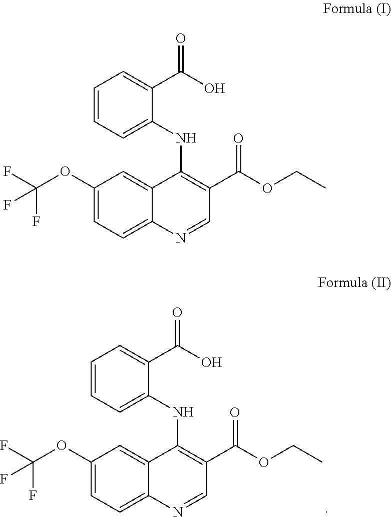 [SMILES: CCOC(=O)c1cnc2ccc(OC(F)(F)F)cc2c1Nc1ccccc1C(=O)O ; CCOC(=O)c1cnc2ccc(OC(F)(F)F)cc2c1Nc1ccccc1C(=O)O]

Embodiment 4. The method of embodiment 1, wherein the composition is administered orally, intranasally, intravenously, intraperitoneally, intramuscularly, intrathecally, intracerebroventricularly, or subcutaneously.

Embodiment 5. The method of embodiment 1, wherein the composition is administered after genetic testing for the presence of a Huntington's disease mutation.

Embodiment 6. The method of embodiment 1, wherein the synaptic defect is a decrease of Synapsin I, Bassoon, Homer1, or any combination thereof.

Embodiment 7. The method of embodiment 1, wherein the subject has Huntington's disease, whereby the administration of the composition treats Huntington's disease or a symptom thereof.

Embodiment 8. The method of embodiment 7, wherein the symptom is uncontrolled movement of the arms, legs, head, face or upper body; a decline in thinking and reasoning skills; or any combination thereof.

Embodiment 9. The method of embodiment 1, wherein the composition is administered after a symptom of Huntington's disease develops in the subject.

Embodiment 10. The method of embodiment 9, wherein the symptom is uncontrolled movement of the arms, legs, head, face or upper body; a decline in thinking and reasoning skills; or any combination thereof.

Embodiment 11. The method of embodiment 1, wherein the composition further comprises a pharmaceutically effective carrier and/or excipient.

### REFERENCES

Aashaq, S., Batool, A., and Andrabi, K. I. (2019). TAK1 mediates convergence of cellular signals for death and survival. Apoptosis 24, 3-20.
Agarwal, V., Bell, G. W., Nam, J. W., and Bartel, D. P. (2015). Predicting effective microRNA target sites in mammalian mRNAs. Elife 4.
Ameres, S. L., Horwich, M. D., Hung, J. H., Xu, J., Ghildiyal, M., Weng, Z., and Zamore, P. D. (2010). Target RNA-directed trimming and tailing of small silencing RNAs. Science 328, 1534-1539.
Boele, J., Persson, H., Shin, J. W., Ishizu, Y., Newie, I. S., Sokilde, R., Hawkins, S. M., Coarfa, C., Ikeda, K., Takayama, K., et al. (2014). PAPD5-mediated 3′ adenylation and subsequent degradation of miR-21 is disrupted in proliferative disease. Proc Natl Acad Sci USA 111, 11467-11472.
Bu, D., Luo, H., Huo, P., Wang, Z., Zhang, S., He, Z., Wu, Y., Zhao, L., Liu, J., Guo, J., et al. (2021). KOBAS-i: intelligent prioritization and exploratory visualization of biological functions for gene enrichment analysis. Nucleic Acids Res 49, W317-w325. Chen, Y., and Wang, X. (2020). miRDB: an online database for prediction of functional microRNA targets. Nucleic Acids Res 48, D127-d131.
Chen, Z. S., Li, L., Peng, S., Chen, F. M., Zhang, Q., An, Y., Lin, X., Li, W., Koon, A. C., Chan, T. F., et al. (2018). Planar cell polarity gene Fuz triggers apoptosis in neurodegenerative disease models. EMBO Rep 19.
Cheng, P. H., Li, C. L., Chang, Y. F., Tsai, S. J., Lai, Y. Y., Chan, A. W., Chen, C. M., and Yang, S. H. (2013). miR-196a ameliorates phenotypes of Huntington disease in cell, transgenic mouse, and induced pluripotent stem cell models. Am J Hum Genet 93, 306-312. de Mezer, M., Wojciechowska, M., Napierala, M., Sobczak, K., and Krzyzosiak, W. J. (2011). Mutant CAG repeats of Huntingtin transcript fold into hairpins, form nuclear foci and are targets for RNA interference. Nucleic Acids Res 39, 3852-3863. Dong, X., and Cong, S. (2019). The Emerging Role of microRNAs in Polyglutamine Diseases. Front Mol Neurosci 12, 156.
Durrenberger, P. F., Fernando, F. S., Magliozzi, R., Kashefi, S. N., Bonnert, T. P., Ferrer, I., Seilhean, D., Nait-Oumesmar, B., Schmitt, A., Gebicke-Haerter, P. J., et al. (2012). Selection of novel reference genes for use in the human central nervous system: a BrainNet Europe Study. Acta Neuropathol 124, 893-903.
Ellis, M. C., O'Neill, E. M., and Rubin, G. M. (1993). Expression of Drosophila glass protein and evidence for negative regulation of its activity in non-neuronal cells by another DNA-binding protein. Development 119, 855-865.
Fukuoka, M., Takahashi, M., Fujita, H., Chiyo, T., Popiel, H. A., Watanabe, S., Furuya, H., Murata, M., Wada, K., Okada, T., et al. (2018). Supplemental Treatment for Huntington's Disease with miR-132 that Is Deficient in Huntington's Disease Brain. Mol Ther Nucleic Acids 11, 79-90.
Garcia-Lopez, A., Monferrer, L., Garcia-Alcover, I., Vicente-Crespo, M., Alvarez-Abril, M. C., and Artero, R. D. (2008). Genetic and chemical modifiers of a CUG toxicity model in Drosophila. PLoS One 3, e1595.
He, Y., and Casaccia-Bonnefil, P. (2008). The Yin and Yang of YY1 in the nervous system. J Neurochem 106, 1493-1502.
Jin, P., Zarnescu, D.C., Zhang, F., Pearson, C. E., Lucchesi, J. C., Moses, K., and Warren, S. T. (2003). RNA-mediated neurodegeneration caused by the fragile X premutation rCGG repeats in Drosophila. Neuron 39, 739-747.
Johnson, R., Zuccato, C., Belyaev, N. D., Guest, D. J., Cattaneo, E., and Buckley, N. J. (2008). A microRNA-based gene dysregulation pathway in Huntington's disease. Neurobiol Dis 29, 438-445.
Jonas, S., and Izaurralde, E. (2015). Towards a molecular understanding of microRNA-mediated gene silencing. Nat Rev Genet 16, 421-433.
Krol, J., Busskamp, V., Markiewicz, I., Stadler, M. B., Ribi, S., Richter, J., Duebel, J., Bicker, S., Fehling, H. J., Schubeler, D., et al. (2010). Characterizing light-regulated retinal microRNAs reveals rapid turnover as a common property of neuronal microRNAs. Cell 141, 618-631.
Lee, S. T., Chu, K., Im, W. S., Yoon, H. J., Im, J. Y., Park, J. E., Park, K. H., Jung, K. H., Lee, S. K., Kim, M., et al. (2011). Altered microRNA regulation in Huntington's disease models. Exp Neurol 227, 172-179.
Li, L. B., Yu, Z., Teng, X., and Bonini, N. M. (2008). RNA toxicity is a component of ataxin-3 degeneration in Drosophila. Nature 453, 1107-1111.
Lieberman, A. P., Shakkottai, V. G., and Albin, R. L. (2019). Polyglutamine Repeats in Neurodegenerative Diseases. Annu Rev Pathol 14, 1-27.
Mangiarini, L., Sathasivam, K., Seller, M., Cozens, B., Harper, A., Hetherington, C., Lawton, M., Trottier, Y., Lehrach, H., Davies, S. W., et al. (1996). Exon 1 of the HD gene with an expanded CAG repeat is sufficient to cause a progressive neurological phenotype in transgenic mice. Cell 87, 493-506.
Marti, E. (2016). RNA toxicity induced by expanded CAG repeats in Huntington's disease. Brain Pathol 26, 779-786.
Marti, E., Pantano, L., Banez-Coronel, M., Llorens, F., Minones-Moyano, E., Porta, S., Sumoy, L., Ferrer, I., and Estivill, X. (2010). A myriad of miRNA variants in control and Huntington's disease brain regions detected by massively parallel sequencing. Nucleic Acids Res 38, 7219-7235.
McCampbell, A., Taylor, J. P., Taye, A. A., Robitschek, J., Li, M., Walcott, J., Merry, D., Chai, Y., Paulson, H., Sobue, G., et al. (2000). CREB-binding protein sequestration by expanded polyglutamine. Hum Mol Genet 9, 2197-2202.
Megret, L., Nair, S. S., Dancourt, J., Aaronson, J., Rosinski, J., and Neri, C. (2020). Combining feature selection and shape analysis uncovers precise rules for miRNA regulation in Huntington's disease mice. BMC Bioinformatics 21, 75.
Messeguer, X., Escudero, R., Farré, D., Núñez, O., Martinez, J., and Albà, M. M. (2002). PROMO: detection of known transcription regulatory elements using species tailored searches. Bioinformatics 18, 333-334.
Nagpal, N., Wang, J., Zeng, J., Lo, E., Moon, D. H., Luk, K., Braun, R. O., Burroughs, L. M., Keel, S. B., Reilly, C., et al. (2020). Small-Molecule PAPD5 Inhibitors Restore Telomerase Activity in Patient Stem Cells. Cell Stem Cell 26, 896-909 e898.
Olmo, I. G., Olmo, R. P., Goncalves, A. N. A., Pires, R. G. W., Marques, J. T., and Ribeiro, F. M. (2021). High-Throughput Sequencing of BACHD Mice Reveals Upregulation of Neuroprotective miRNAs at the Pre-Symptomatic Stage of Huntington's Disease. ASN Neuro 13, 17590914211009857.
Omori, E., Inagaki, M., Mishina, Y., Matsumoto, K., and Ninomiya-Tsuji, J. (2012). Epithelial transforming growth factor beta-activated kinase 1 (TAK1) is activated through two independent mechanisms and regulates reactive oxygen species. Proc Natl Acad Sci USA 109, 3365-3370.
Omori, E., Matsumoto, K., Sanjo, H., Sato, S., Akira, S., Smart, R. C., and NinomiyaTsuji, J. (2006). TAK1 is a master regulator of epidermal homeostasis involving skin inflammation and apoptosis. J Biol Chem 281, 19610-19617.
Peng, S., Guo, P., Lin, X., An, Y., Sze, K. H., Lau, M. H. Y., Chen, Z. S., Wang, Q., Li, W., Sun, J. K., et al. (2021). CAG RNAs induce DNA damage and apoptosis by silencing NUDT16 expression in polyglutamine degeneration. Proc Natl Acad Sci USA 118.
Schneider, C. A., Rasband, W. S., and Eliceiri, K. W. (2012). NIH Image to ImageJ: 25 years of image analysis. Nat Methods 9, 671-675.
Shi, H., Shen, H., Xu, J., Zhao, S., Yao, S., and Jiang, N. (2018). MiR-143-3p suppresses the progression of ovarian cancer. Am J Transl Res 10, 866-874. Shi, Y., Seto, E., Chang, L. S., and Shenk, T. (1991). Transcriptional repression by YY1, a human GLI-Kruppel-related protein, and relief of repression by adenovirus E1A protein. Cell 67, 377-388.
Shibuya, H., Yamaguchi, K., Shirakabe, K., Tonegawa, A., Gotoh, Y., Ueno, N., Irie, K., Nishida, E., and Matsumoto, K. (1996). TAB1: an activator of the TAK1 MAPKKK in TGF-beta signal transduction. Science 272, 1179-1182.
Steffan, J. S., Bodai, L., Pallos, J., Poelman, M., McCampbell, A., Apostol, B. L., Kazantsev, A., Schmidt, E., Zhu, Y. Z., Greenwald, M., et al. (2001). Histone deacetylase inhibitors arrest polyglutamine-dependent neurodegeneration in Drosophila. Nature 413, 739-743.
Takaesu, G., Kishida, S., Hiyama, A., Yamaguchi, K., Shibuya, H., Irie, K., NinomiyaTsuji, J., and Matsumoto, K. (2000). TAB2, a novel adaptor protein, mediates activation of TAK1 MAPKKK by linking TAK1 to TRAF6 in the IL-1 signal transduction pathway. Mol Cell 5, 649-658.
Thomas-Chollier, M., Hufton, A., Heinig, M., O'Keeffe, S., Masri, N. E., Roider, H. G., Manke, T., and Vingron, M. (2011). Transcription factor binding predictions using TRAP for the analysis of ChIP-seq data and regulatory SNPs. Nat Protoc 6, 18601869.
Tsoi, H., Lau, C. K., Lau, K. F., and Chan, H. Y. (2011). Perturbation of U2AF65/NXF1 mediated RNA nuclear export enhances RNA toxicity in polyQ diseases. Hum Mol Genet 20, 3787-3797.
Tsoi, H., Lau, T. C., Tsang, S. Y., Lau, K. F., and Chan, H. Y. (2012). CAG expansion induces nucleolar stress in polyglutamine diseases. Proc Natl Acad Sci USA 109, 13428-13433.
Tu, H., Chen, D., Cai, C., Du, Q., Lin, H., Pan, T., Sheng, L., Xu, Y., Teng, T., Tu, J., et al. (2020). microRNA-143-3p attenuated development of hepatic fibrosis in autoimmune hepatitis through regulation of TAK1 phosphorylation. J Cell Mol Med 24, 1256-1267.
Urbanek, M. O., and Krzyzosiak, W. J. (2016). RNA FISH for detecting expanded repeats in human diseases. Methods 98, 115-123.
Wang, X., Destrument, A., and Tournier, C. (2007). Physiological roles of MKK4 and MKK7: insights from animal models. Biochim Biophys Acta 1773, 1349-1357. Wang, Z. M., Dong, X. Y., and Cong, S. Y. (2020). Bioinformatic analysis of a microRNA regulatory network in Huntington's disease. J Integr Neurosci 19, 641-650.
Warrick, J. M., Morabito, L. M., Bilen, J., Gordesky-Gold, B., Faust, L. Z., Paulson, H. L., and Bonini, N. M. (2005). Ataxin-3 suppresses polyglutamine neurodegeneration in Drosophila by a ubiquitin-associated mechanism. Mol Cell 18, 37-48.
Wong, S. L., Chan, W. M., and Chan, H. Y. (2008). Sodium dodecyl sulfate-insoluble oligomers are involved in polyglutamine degeneration. Faseb j 22, 3348-3357. Wyman, S. K., Knouf, E. C., Parkin, R. K., Fritz, B. R., Lin, D. W., Dennis, L. M., Krouse, M. A., Webster, P. J., and Tewari, M. (2011). Post-transcriptional generation of miRNA variants by multiple nucleotidyl transferases contributes to miRNA transcriptome complexity. Genome Res 21, 1450-1461.
Yang, X., Kovalenko, D., Nadeau, R. J., Harkins, L. K., Mitchell, J., Zubanova, O., Chen, P. Y., and Friesel, R. (2004). Sef interacts with TAK1 and mediates JNK activation and apoptosis. J Biol Chem 279, 38099-38102.
Zhang, D., Hu, Y., Sun, Q., Zhao, J., Cong, Z., Liu, H., Zhou, M., Li, K., and Hang, C. (2013). Inhibition of transforming growth factor beta-activated kinase 1 confers neuroprotection after traumatic brain injury in rats. Neuroscience 238, 209-217. Zhang, N., and Ashizawa, T. (2017). RNA toxicity and foci formation in microsatellite expansion diseases. Curr Opin Genet Dev 44, 17-29.
Zhang, Y. E. (2017). Non-Smad Signaling Pathways of the TGF-beta Family. Cold Spring Harb Perspect Biol 9.
Zucchelli, S., Marcuzzi, F., Codrich, M., Agostoni, E., Vilotti, S., Biagioli, M., Pinto, M., Carnemolla, A., Santoro, C., Gustincich, S., et al. (2011). Tumor necrosis factor receptor-associated factor 6 (TRAF6) associates with huntingtin protein and promotes its atypical ubiquitination to enhance aggregate formation. J Biol Chem 286, 2510825117.

We claim:

1. A method of reducing cell death; reducing phosphorylation levels of TAK1, MKK4, or JNK; suppressing caspase 3 cleavage; or rescuing a synaptic defect in a subject; or any combination thereof, comprising:
administering an effective amount of a composition comprising a Poly(A) RNA polymerase D5 (PAPD5) small molecule inhibitor to the subject.

2. The method of claim 1, wherein the PAPD5 small molecule inhibitor is derived from quinolone.

3. The method of claim 1, wherein the PAPD5 small molecule inhibitor is BCH001, according to formula (I) or RG7834, according to formula (II):

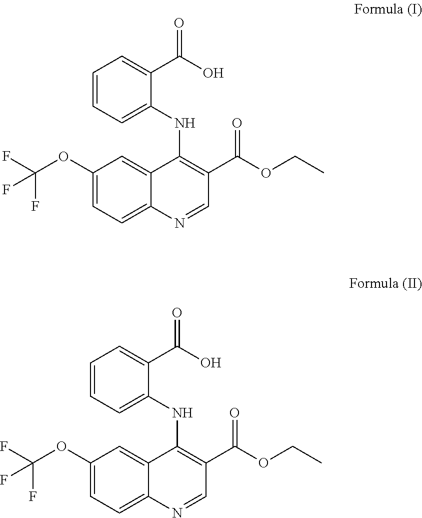 [SMILES: CCOC(=O)c1cnc2ccc(OC(F)(F)F)cc2c1Nc1ccccc1C(=O)O ; CCOC(=O)c1cnc2ccc(OC(F)(F)F)cc2c1Nc1ccccc1C(=O)O]

4. The method of claim 1, wherein the composition is administered orally, intranasally, intravenously, intraperitoneally, intramuscularly, intrathecally, intracerebroventricularly, or subcutaneously.

5. The method of claim 1, wherein the composition is administered after genetic testing for the presence of a Huntington's disease mutation.

6. The method of claim 1, wherein the synaptic defect is a decrease of Synapsin I, Bassoon, Homer1, or any combination thereof.

7. The method of claim 1, wherein the subject has Huntington's disease, whereby the administration of the composition treats Huntington's disease or a symptom thereof.

8. The method of claim 7, wherein the symptom is uncontrolled movement of the arms, legs, head, face, or upper body; a decline in thinking and reasoning skills; or any combination thereof.

9. The method of claim 1, wherein the composition is administered after a symptom of Huntington's disease develops in the subject.

10. The method of claim 9, wherein the symptom is uncontrolled movement of the arms, legs, head, face, or upper body; a decline in thinking and reasoning skills; or any combination thereof.

11. The method of claim 1, wherein the composition further comprises a pharmaceutically effective carrier and/or excipient.
